(19) United States
(12) Patent Application Publication
(10) Pub. No.: US 2025/0185930 A1
(43) Pub. Date: Jun. 12, 2025

(54) Holistic and Sustainable Computing Framework for AI-Enabled Health Applications

(71) Applicant: Board of Regents, The University of Texas System, Austin, TX (US)

(72) Inventors: Dakai Zhu, San Antonio, TX (US); Amanda Fernandez, San Antonio, TX (US); Jianwei Niu, San Antonio, TX (US); Rocky Slavin, San Antonio, TX (US); Wei Wang, San Antonio, TX (US); Mimi Xie, San Antonio, TX (US)

(21) Appl. No.: 18/976,134

(22) Filed: Dec. 10, 2024

Related U.S. Application Data
(60) Provisional application No. 63/608,297, filed on Dec. 10, 2023.

(51) Int. Cl. A61B 5/024

(52) U.S. Cl. CPC A61B 5/02416

(57) ABSTRACT

Certain aspects are directed to methods for estimating heart rate (HR) with high accuracy (i) obtaining signal from one or more photoplethysmography (PPG) sensor in contact with a subject, (ii) combining (a) signal processing generating first HR estimations and (b) passing the first HR estimations through machine learning (ML) model generating second accurate HR estimations reducing the PPG sampling frequency to about or less than 25 Hz and providing higher HR estimation accuracy achieving less than 5% mean average prediction errors (MAPE).

### RELATED APPLICATIONS

This application is a US Utility application claiming priority to U.S. Provisional Application 63/608,297 filed Dec. 10, 2023 which is incorporated herein by reference in its entirety.

### FIELD

Aspects of the invention(s) described herein are generally directed to medicine and patient monitoring, in particular heart rate monitoring.

### BACKGROUND

ΔI-enabled health applications are widely adopted today to support the caring of senior people and patients with chronic diseases. The computing infrastructure for these health applications usually involves a myriad of hardware and software, including wearable sensors, embedded devices, edge servers, and the cloud. Current research on AI-enabled health applications primarily focused on their functionalities and related models/algorithms. However, there lacks research on the usability of these applications, especially on supporting the long-term sustainable deployments of these applications, which demands both energy-efficient and cost-effective computing infrastructures.

Improving the sustainability in computing infrastructure for health applications requires the knowledge of multiple areas in computer systems, such as sensing devices, embedded systems, and edge/cloud computing, where most health professionals do not have such knowledge. With a team of computer scientists, AI experts, and health professionals, this project aims at developing automated optimization frameworks to improve energy- and cost-efficiency of the computing infrastructure of AI-enabled health applications. Three research trusts are proposed to build inter-related optimization frameworks to: (a) reduce energy consumption of sensing devices; (b) minimize the cost of cloud deployment; and (c) optimize the energy and cost of the overall infrastructure by leveraging edge servers. The proposed frameworks only require limited inputs from health professionals and AI experts regarding the sensing/computing needs, so that people without computer system background can also easily optimize the design of their AI health systems. An in-home glucose monitoring prototype system for patients with diabetes will be built to validate and evaluate the proposed frameworks.

### SUMMARY

Heart rate (HR) is an important vital sign for the cardiovascular system and has been widely used as a biomarker for diagnostic and early prognostic of several diseases such as hypertension and heart failure. Besides the critical condition monitoring in the hospital setting, many applications also depend on continuously measured HR, such as fitness tracking, biometric identification, and frailty detection. Therefore, it is desirable to have a real-time HR monitoring system, which can conveniently provide accurate data in an effective manner to support such applications.

Scientific contributions described herein are in both computing and health domains. The first one is the energy/cost-efficiency design and optimization framework for embedded sensing devices. To support sustainable and long-term operation of such devices, the hardware and software design and optimization framework will explore the energy and latency models for a wide range of sensors, processors, memory, and energy harvesters; where both non-volatile memory and energy harvesting technologies will be exploited. This second contribution is the cost-efficiency optimization framework for cloud AI model deployments. By employing novel cloud performance testing techniques, this framework finds the lowest cost cloud resource configurations using only the AI model itself, without the needs of its user to provide the resource usage information. The third contribution will leverage edge computing to integrate sensing devices, edge servers, and cloud systems, where various offloading models/algorithms will be investigated to combine the above two frameworks for designing a holistic optimized AI health computing infrastructure. The last contribution comes from the case study and prototype of an in-home glucose monitoring health system for patients with diabetes.

Certain embodiments are directed to methods for estimating heart rate (HR) with high accuracy comprising: (i) obtaining signal from one or more photoplethysmography (PPG) sensor in contact with a subject, (ii) combining (a) signal processing generating first HR estimations and (b) passing the first HR estimations through machine learning (ML) model generating second accurate HR estimations reducing the PPG sampling frequency to about or less than 25 Hz and providing higher HR estimation accuracy achieving less than 5% mean average prediction errors (MAPE). The ML can be selected from Decision Tree (DT), Random Forest (RF), K-nearest neighbor (KNN), Support vector machines (SVM), and Multi-layer perceptron (MLP), or other known ML. In certain aspects the ML is DT. The DT can have 10 to 20 input features. The ML models, e.g., DT model, can have a model size of about or less than 10 KB and a shorter inference time of less than 3 microseconds (μs). In certain aspects the PPG is located on the skin of a subject. In a further aspect the PPG is placed on a subject's finger, wrist, or earlobe. Certain embodiments are directed to a device or wearable device for estimating heart rate (HR) with high accuracy configured to (i) obtain signal from one or more photoplethysmography (PPG) sensor in contact with a subject, (ii) combine signal processing and machine learning (ML) reducing the PPG sampling frequency to about or less than 25 Hz and providing higher HR estimation accuracy. The ML can be selected from Decision Tree (DT), Random Forest (RF), K-nearest neighbor (KNN), Support vector machines (SVM), and Multi-layer perceptron (MLP). In certain aspects the ML is DT. The ML, e.g., DT, can have 10 to 20 input features. The wearable can be configured to contact the PPG with skin of a subject. In certain aspects the wearable device is configured to be placed on a subject's finger, wrist, or earlobe when in use.

Photoplethysmography (PPG) is a non-invasive optical technique used to detect blood volume changes in the microvascular bed of tissue. PPG works by shining light into the skin and then measuring the amount of light either transmitted or reflected back. The light is typically in the green or infrared spectrum. Blood absorbs light more than surrounding tissues, so changes in blood flow cause variations in the amount of light detected by a photodetector. The most common application of PPG is in pulse oximeters, where the changes in light absorption are used to calculate the oxygen saturation level in the blood (SpO2) and the heart rate. PPG can detect the pulsatile component (AC), which corresponds to the heartbeat, superimposed on a slowly varying baseline (DC), which reflects the overall blood volume. Applications include: (i) Vital Signs Monitoring: Heart rate, heart rate variability, respiratory rate can be monitored via PPG. (ii) Sleep Analysis: PPG can be used in wearable devices to monitor sleep quality by detecting changes in heart rate and blood oxygen levels. (iii) Vascular Assessment: It can assess conditions like peripheral arterial disease by measuring blood flow changes. (iv) Fitness Trackers: Many wearable devices use PPG sensors to track fitness metrics.

The PPG waveform consists of a systolic peak (the peak of the waveform corresponding to the arterial pressure wave), a dicrotic notch (a small notch on the downslope of the waveform, representing the closure of the aortic valve), diastolic phase (the remainder of the waveform until the next cycle begins). PPG has become integral in both medical and consumer health devices due to its simplicity, cost-effectiveness, and the non-invasive nature of the technology.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Overview of the common computing infrastructure for AI-enabled applications.

FIG. 2. Architecture of typical health sensing devices.

FIG. 3. Average prediction errors for the original BO regression and PT3Cloud-enhanced BO regression.

FIG. 4. Optimizations: model optimization, edge offloading, and cloud resource selection.

FIG. 5. Data pipeline of real-time on-device HRV monitoring system.

FIG. 6. Shows the setup of the demonstration. The PPG sensor is MAXREFDES117 that provides reliable light signals and has noise filter units. The MSP430FR5994 development kit is one of the MSP430 families produced by Texas Instruments.

FIG. 7. Illustration of system design and experiment flow.

FIG. 8. The PPG HRV, ECG HRV, and ML predicted HRV in the sleep scenario when k=300 and the ML algorithm is RF. The RF predicted HRV is closer to the ECG HRV compared with PPG HRV.

FIG. 9. MAPE for PPG HRV and ML predicted HRV for different ML algorithms and scenarios when k=30. The PPG HRV has a higher error than ML predicted HRV for all ML algorithms and all scenarios.

FIG. 10A-10B. (A) ML model size for different k−the number of PPG HR in ML features in sit scenario. DT, MLP, and RF have a smaller model size while KNN and SVM have a large model size. (B) MAPE for different k−the number of PPG HR in ML in sleep scenario. A larger k contributes to higher prediction accuracy and all ML predicted HRV have a MAPE smaller than 20%.

FIG. 11. Overview of framework design.

FIG. 12. Illustration of low power set up including sensor and board.

FIG. 13. Pipeline for real-time HR/HRV monitoring.

FIG. 14. Power analysis modeling.

FIG. 15. Runtime adaptive configurations.

FIG. 16. CPU cycle in preprocessing plotted against input size.

FIG. 17. CPU cycle in inference plotted against MAC operations.

FIG. 18. The effect of model size on model accuracy.

FIG. 19. Graphic of battery life plotted against sampling rate.

FIG. 20. Graphic of battery life plotted against SMCLK.

FIG. 22. Graphic of battery life plotted against DCO.

FIG. 23. The architecture of the PPG-based HR monitoring system.

FIG. 24. Major steps of the PPG-Based HR monitoring system.

FIG. 25. Connections between Pi and sensors.

FIG. 26. HR estimation MAPE for different subjects in the ISPC data set (MLP model's feature size is 2). “MLP” stands for our method that combines signal processing and MLP, and “Sig-proc” stands for the signal-processing-only method reproduced based on the ISPC paper (Zhang et al. IEEE Transactions on biomedical engineering, 62 (2): 522-31, 2014).

FIG. 27. HR estimation trace for subject 11 in the ISPC data set (MLP model's feature size is 2).

FIG. 28. Model sizes for different ML models with different number of features when estimating ISPC HR.

FIG. 29. Model inference time for each HR reading with different ML models and number of features when estimating ISPC HR.

FIG. 30. HR estimation MAPEs for different scenarios and ML algorithms using our data set (MLP model's feature size is 10).

FIG. 31. Partial testing set HR estimation trace for the daily activities scenario (MLP model's feature size is 10). “Sig-proc” stands for the signal-processing only method.

FIG. 32. HR estimation MAPE for different number of features with our collected daily scenario data.

FIG. 33. ML model size for different number of features when estimating HR using our collected daily scenario data.

FIG. 34. ML inference time for different number of features when estimating HR using our collected daily scenario data.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

AI-enabled health applications have been widely adopted today to support the caring of senior citizens and patients with chronic diseases. These health applications typically rely on a variety of sensors (such as pulse oximeter and motion sensors) and AI models to infer the physical or mental conditions of the person being cared for. The inferred physical and mental conditions may be used to provide feedback to the patients, assist the diagnosis, or notify the health providers in case of emergency.

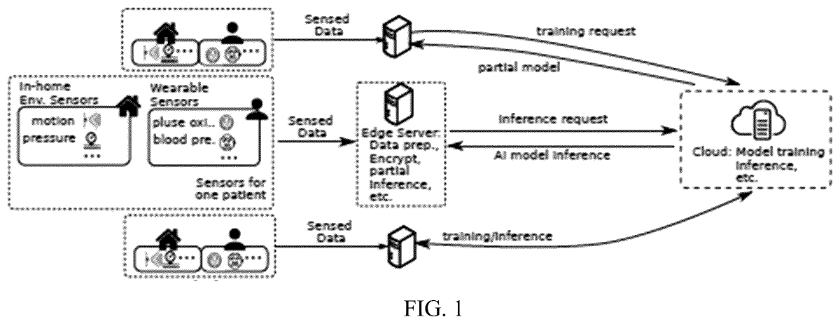

To support the computing requirements of the AI-enabled health applications, their information infrastructures usually involve a myriad of computing devices, where FIG. 1 gives an overview of such common computing infrastructures. As FIG. 1 shows, these computing infrastructures are usually multi-layered. The front layer is usually composed of various wearable and environmental sensors, such as accelerometer, temperature sensor, pulse oximeter, and pressure sensors, which provide the data as inputs to various AI models to make inferences (Chen et al., IEEE Engineering in Medicine and Biology 27th Annual Conference, pages 3551-3554, 2005; Chiang and Dey, IEEE 20th International Conference on e-Health Networking, Applications and Services (Healthcom), 2018; Thierry, IntechOpen, 2019). Such sensors cannot operate by themselves and are usually attached to wearable or non-wearable embedded sensing devices, which have computing and other supporting components, such as processors, memory storage, and power modules. The backend computing layer is usually the cloud, which executes AI models (i.e., making inferences) and supports the continuous training of the AI models (Mark Bishop Ring. Continual Learning in Reinforcement Environments. PhD thesis, University of Texas at Austin, 1994). In the middle layer, edge servers can be employed to conduct data pre-processing (e.g., data aggregation or anonymization) tasks that are too computationally intensive for embedded devices (Alameddine et al., IEEE Journal on Selected Areas in Communications, 37 (3): 668-682, 2019; Li et al., Journal of Parallel and Distributed Computing, 123:69-76, 2019). Moreover, edge servers may also assist AI inference and reduce inference latency by executing partial layers of large AI models or even completely execute small AI models for cost e ciency (Kang et al., Proceedings of the Twenty-Second International Conference on Architectural Support for Programming Languages and Operating Systems, 2017).

Most prior AI-enabled health research studies primarily focus on their functionality and related AI models/algorithms. However, there lacks research on the usability of these applications, especially on supporting the long-term sustainable deployments of these applications with both energy-efficient and cost-efficient computing infrastructures.

Energy-efficiency has been a major design constraint for AI-enable health applications. Wearable sensing devices typically operate on battery power, and their energy efficiency directly affects the operational time of these devices. Environmental sensors may be also placed at locations in a house without easy access to power outlets, making it necessary for them to operate on batteries. For mobile sensors deployed for elder care, energy-efficiency is even more important, as elder people may be more likely to forget to recharge or change the battery of their sensing devices. Furthermore, with modern energy harvesting technology, the operational time of a properly designed sensing device may be considerably extended with better use of energy sources. In the ideal case, a sensing device with energy harvesting may operate without being charged by its user. Therefore, it is important to design the sensing devices to operate with low or little power consumption, tolerate intermittent power supply and energy loss, and/or be able to harvest energy from the environment.

For the large-scale deployment of AI-enabled health applications to serve hundreds or even thousands of patients simultaneously, it must be designed with high cost-efficiency for deployment and operation. Many chronic health issues, such as Type 2 diabetes mellitus (T2DM), become major public health concerns as they are burdensome for individuals, health systems, and society. Hence, the computing infrastructure for AI-enabled health applications should not further exacerbate this burden. However, machine-learning models, especially personalized models, may have a high demand for computing resources, which, in turn, incurs high cost for system operation. Therefore, it is also important to design the multi-layered computing infrastructures to operate with low cost so that the AI-powered health applications can reach the low-income communities.

There have been research studies that optimize the designs for better energy- and cost-efficiency in wireless sensors, embedded systems or edge/cloud computing. However, the solutions proposed in these research studies cannot be easily employed by health application developers (i.e., health professionals, AI experts, and software engineers) as they usually do not have the required knowledge in computer systems. Additionally, there is very limited prior work that provides solutions to determine the energy and cost efficient designs for a multi-layered and multi-modal system for a given specific health problem, which demands a holistic evaluation of various design options for different layers and AI models of the connected system, from the applications, sensors, edge servers to the clouds

In this proposed project, our main goal is to provide effective and automated system optimization frameworks for multi-layered AI health systems that can be utilized by health application developers without requiring any prior knowledge of computer systems. To achieve this goal, we propose three research thrusts: (1) a design and optimization framework for sustainable embedded sensing devices powered with energy harvesting techniques; (2) an automated optimization framework for cost-efficiency cloud AI deployments; and (3) edge-enabled optimizations of multi-layer computing infrastructure design for improved sustainability and cost-efficiency. The proposed optimization frameworks will be evaluated through extensive simulations with publicly available AI-enabled health applications on their energy and cost benefits. Moreover, the frameworks will be validated through building a prototype diabetes management system, which employs various environmental and wearable sensing devices to predict the future blood glucose levels for T2DM patients to help them plan their daily activities and diets.

AI-enabled Health Applications and Diabetes Management: The advancements in sensing and computing techniques enabled machine learning techniques to predict various health conditions of patients based on an ever-growing amount of data. With the growing of elder population, where many may suffer from chronic diseases, including type 2 diabetes mellitus (T2DM), such AI-enabled health applications and systems provide pivotal technical supports for them to live independently. Powered by the computation capabilities in clouds and various sensing devices, several AI-enabled health systems have been developed to provide health monitoring for various scenarios (including elder caring). For instance, CoCaMAAL is an ambient assisted living system that combines biomedical sensors and cloud computing to support machine learning computations.

With many people suffering from diabetes mellitus—a serious chronic disease, research studies have been conducted to help diabetes treatment with AI and machine learning techniques. For instance, Plis et al. proposed a generic physiological model of blood glucose dynamics to extract informative features to train a support vector regression model on patient-specific data. As the relation between input features and glucose levels is nonlinear, dynamic, interactive, and patient-specific, nonlinear regression models were used to build the predictive models. Furthermore, neural networks have increasingly been used to model glucose levels, including multilayer perceptrons, recurrent neural networks, and convolutional recurrent neural networks. In our prior work, we have developed Long-Short-Term-Memory (LSTM) based glucose level predictive personalized models, which employed features including patient self-reported diets and physical activities, as well as previous glucose levels obtained from a blood glucometer. With the help of wearable sensors and environmental sensors, we have been working to improve such models without error-prone self-reporting from the patients.

However, the existing studies on AI-enabled health applications and systems focused primarily on the functionalities of the developed AI models/algorithms. To the best of our knowledge, there is no prior work for sustainable system designs that holistically optimizes the machine-learning models, edge/cloud coordination and deployments. Therefore, additional research needs to be conducted to investigate how to holistically optimize multiple layers of the system while considering the actual sensing and computing requirements of various health applications to improve their energy and cost efficiencies.

Energy Efficiency and Sensing Devices: With technology advancements in sensing and wireless communication (e.g., BLE, ZigBee or WiFi), sensing devices with limited on-board computation (e.g., min/max operations) can be accessed remotely to support different smart applications, including AI-enabled health applications. While it may be possible to connect some sensing devices to wall power in certain settings, many may still need a battery for easy deployment. For battery-powered sensing devices, it is crucial to improve their energy efficiency. Many studies have been reported on managing energy efficiency of such sensing devices using various low-power techniques. Moreover, to support extended operation duration, sensing devices may be equipped with various energy-harvesting (EH) components to get energy from ambient environment (e.g., solar or vibration). One of the key issues for such energy-harvesting devices is how to tackle the intermittent power supply due to unstable energy sources. Based on nonvolatile memory (NVM), researchers have proposed various techniques (such as checkpointing) to store intermediate results to NVM in case of power failure. In addition, several studies reported a processor design with ferroelectric non-volatile registers that have the access efficiency close to that of SRAM with superior endurance. Such non-volatile processors are ideal for energy-harvesting devices with their ability to more efficiently store the intermediate running states. Nonetheless, the most efficient sensing device design requires properly coordinating software and hardware, and should be tailored according to the actual application's needs (such as sampling frequencies).

Cost-Efficient Cloud Deployments: As clouds usually offer a large number of options for resource allocation, determining the best-performing and/or lowest-cost resource allocation scheme is an active research topic. Using the internal structure of a cloud application, Ernest predicted the performance of a cloud application when it was executed with a resource allocation. PARIS predicted the performance of a cloud application utilizing its system-level resource usage information, such as the memory usage and CPU utilization. Arrow employs Bayesian Optimization to search for the optimal resource allocation with the information of low-level system resource usages. Hsu et al. proposed employing multi-armed bandit algorithm to find the optimal resource allocation for applications with similar behaviors. Other studies also grouped applications with similar system-level behaviors and built performance prediction models for each group of applications by using some of these applications as training benchmarks. However, these studies require collecting information that was not easily accessible to non-cloud experts, such as application's internal program structure, low-level system resource usages, and similarly-behaving applications.

Cherrypick was a completely-automated optimization framework that search for the optimal resource allocation using Bayesian Optimization. Cherrypick did not require any additional information other than the cloud application and its input data. Our proposed cloud optimization framework is inspired by Cherrypick. However, as shown later, the accuracy and effectiveness of Cherrypick are significantly affected by the reliability of its training data. Therefore, in this project, we will extend Cherrypick with our prior work on cloud performance testing to design a more reliable and accurate cloud resource allocation optimization framework for health professionals and AI experts.

Edge Computing and Offloading: It is not until recently edge computing emerged to address the issues with tremendous amount of data transmitted in networks following the principle of processing them at the proximity of data sources. Edge servers, which include any computing (and network) resources along the path between data sources and clouds, play a crucial role in improving energy efficiency as well as enhancing data security and privacy. The key technique that enables these benefits is offloading, which includes two important aspects. First, relatively complex computation can be moved to dedicated edge servers from embedded devices to improve their energy efficiency. Second, edge servers may also help learning oriented tasks with model partitioning to improve the cost efficiency of smart applications deployed on clouds. While there have been many research studies on offloading to improve various aspects of system efficiency in different settings, there were only limited studies that considered all computing components. Clearly, a holistic optimization framework need to incorporate the energy and cost efficiency all involved components, including sensing devices, edge servers and clouds.

The overview of a common computing infrastructure for AI-enabled health application was shown in FIG. 1. We envision that such a system typically includes the following components: a set of sensors (including both environmental and wearable) for data collection and preprocessing, edge servers for data preprocessing and partial model inference, and a back-end on the cloud for model training, model inference, data storage and data query. As stated previously, this project involves three research tasks.

Thrust 1 focuses on designing the automated optimization framework to improve the energy-efficiency of embedded sensing devices. Thrust 2 focuses on designing the automated optimization framework to improve the cost-efficiency of the cloud deployments. Thrust 3 combines the optimization frameworks from Thrusts 1 and 2 to explore offloading computations to the edge servers to further improve energy efficiency and cost-efficiency. The rest of this section describes each research thrust in detail.

### Thrust 1: Design and Optimizations for Sustainable Sensing Devices

Thrust 1 focuses on developing an automated design and optimization framework for energy-efficient embedded sensing devices. The energy consumption and cost of a sensing device can be significantly reduced if both the designs of its software and hardware are tailored to the specific requirements of a health application, including the types of sensors, data collection frequency, and data processing workload. However, the design of the sensing devices also requires additional hardware information such as the energy and performance of the required sensors, processors, memory, and energy sources which are generally not accessible to health application developers. Specifically, in Thrust 1, we will model the energy consumption, performance and cost of various hardware and software components, including processors, non-volatile memory, energy harvesting technologies, and low-power communication protocols, needed by the sensing devices of AI-enabled health applications. Then, based on the input from the health professionals and AI experts on the specific requirements of health applications, energy/cost-oriented designs and optimizations will be investigated for energy-harvesting sensing devices to meet different computation and communication requirements. The developed optimization framework will generate the low-cost design for the required sustainable sensing devices with the best energy efficiency.

### Task 1-1: Hardware Design and Optimizations for Customized Sensing Devices.

Given a set of sensing and performance requirements as well as other constraints (e.g., size, weight and cost) of a specific health application, this sub-task aims to provide an optimized hardware design option for the customized sensing device that supports sustainable operations. With the emerging nonvolatile memory (NVM) and energy harvesting being considered, the general hardware architecture for sensing devices is shown in FIG. 2, where a set of exemplary sensors for a possible health monitoring device are illustrated. Energy storage (e.g., a rechargeable battery or capacitor) is also included in addition to a power regulator that is used to maintain a constant operating voltage level and regular I/O interfaces. The key hardware design choices that play an important role in energy efficiency and performance include the type of sensors, processor, memory size, transceiver, energy harvester and energy storage capacity. Such design choices also need to consider the challenge of integrating all the selected hardware components under the specified constraints (e.g., size, weight and cost).

Sensors: the types and numbers of sensors are determined based on the kinds of data required by a given health application. We will evaluate the accuracy, performance, and energy consumption of available sensors on market and integrate a group of them that can collect adequate data without interference for the given health application. FIG. 2 illustrates an example set of sensors for glucose level monitoring.

Processors and memory: We will model the required computation power of processor and memory size for software and data storage according to the requirements of the given health application. To address the intermittent energy issues, we will exploit non-volatile processor (NVP) by integrating emerging NVM (FRAM, ReRAM, Flash) as on-chip memory due to their features of non-volatility, low power, and high density. In case of power failure, the system state can be saved into the on-chip NVM efficiently for fast resumption. For low-power scenario, dynamic voltage and frequency scaling (DVFS) enabled processors will be considered to adjust the operating mode according to the available energy. In addition to data fusion, compression and filtering aiming at reducing data storage and communication cost, encryption may be needed for privacy consideration, especially with the health-related data. Based on the sensing (e.g., sampling rate), data processing requirements, as well as constraints such as cost, we will study optimization strategies to translate these features into energy efficient design choices for the type and size of processors and memory.

Energy harvesters: We will explore different energy harvesting technologies considering their characteristics such as predictability, controllability, and magnitude, as well as the capacity of the energy storage device to enable sustainable functioning sensing devices. We will consider solar energy, indoor light, and RF energy, as well as ultra low power resources, such as micro-solar, breathing (0.42 W), and body heat (2.4˜4.8 W) which could provide sufficient power to drive the devices at low duty cycles. The objective is to provide enough energy for the sensing devices to satisfy the needs of health applications within the size and cost constraint.

For transceivers, different transmission technologies (such as Bluetooth, ZigBee and LP WiFi) support different transmission ranges, bit rate, throughput, and frequency at different power consumption. Based on the data transmission needs, we will select and integrate the low-power transmission technology (e.g., Bluetooth or ZigBee) into the sensing device that satisfy the need of health applications.

Task 1-2: Software Optimizations for Sensing Devices with Resource Constraints. In this sub-task, with the hardware designs for sensing devices from Task 1-1, we will study software optimization techniques to manage the sensing tasks with the hardware resources and limited/intermittent energy supply. In particular, we will design several software components for energy-harvesting sensing devices, including voltage monitor, checkpoint handler, event handler, frequency modulator, and I/O management to support efficient data collection with the needed data sampling rate, required computation, and demanded communication. Here, the first three components are responsible for the energy harvesting system support. Specifically, the voltage monitor module is responsible for proactively initiating voltage detection and voltage analysis based on which we can predict the energy harvesting efficiency. The checkpoint handler is responsible for saving the necessary system state when there is a power failure. The event handler is responsible for waking up the system and putting the system to sleep mode when necessary by selecting reasonable wake-up and backup voltage thresholds.

Low-power Operation Management: The sampling rate of the sensors, operating frequency of the microcontroller units (MCU), and transmission rate are the key factors to be coordinated for the health application. These parameters can result in different energy consumption levels of sensing devices. Clearly, there is a trade-off between energy consumption and performance. For example, while a higher sampling rate can provide more data, a high operating frequency of the MCU will be required to process the data and a larger NVM is also required to buffer them. However, a large NVM and high CPU frequency mean more energy consumption. If there is not sufficient energy in the energy buffer or the energy harvesting efficiency is low, we should reduce the sampling rate and CPU frequency to let the wearable device operate with ultra-low power. We can also reduce or even stop the data transmission according to the energy availability. Here, the frequency modulator and I/O management components will decide the proper operating parameters during run time.

Energy-aware Task Scheduler: We will also design an energy-aware task scheduler to schedule different types of tasks based on the sensing needs of health application and available energy budget. Although checkpoint can be employed to suspend a task during a power failure, not all tasks (such as sensing and transmission tasks) can be checkpointed. After a power failure, such tasks will have to restart which can significantly increase the latency and energy consumption. Therefore, we need a scheduler to prioritize them to complete them first before a power outage. Moreover, we should make a trade-off between the design parameters (such as battery capacity) and the energy harvesting efficiency to reduce the number of power failures. The voltage monitor module can proactively initiate voltage detection and analysis. Specifically, it will study the voltage on the buffer battery/capacitor since it reflects the actual power supply of the energy harvester. If the voltage on the storage capacitor drops, the energy harvester generates less power than consumed. Based on the history voltages and variation rate, we will design an energy predictor to predict the amount of energy that can be harvested in the considered time period and make scheduling decisions appropriately.

Task 1-3: Hardware/Software Co-design and Optimizations and Its Application The above two sub-tasks focus on the hardware and software aspects separately. In this third sub-task, we will further explore system optimizations with hardware/software co-design approaches. Given that certain processing (such as data fusion, compression or encryption) can be either performed with special hardware (e.g., accelerators) or implemented in software, there will be a clear trade-off between cost and performance. Moreover, with carefully designed scheduling algorithms, the requirement for battery capacity may be reduced to support certain operation requirements, especially with the features (e.g., power traces) of energy harvesting being considered. We may also reduce the required size of NVM, if software design can reduce data size. In particular, the co-design optimization approaches will be utilized to develop a prototype wearable sensing device with multiple related sensors while considering the sensing requirements (e.g., sample rates and data amount) for glucose monitoring, which will facilitate the case study on diabetes management to validate the proposed framework.

### Thrust 2: Cost-Efficiency Cloud Deployments

To support continuous model training/updating and the inference of large AI models, cloud computing is typically employed as the backend of AI-enabled health applications, because clouds provide a large amount of on-demand computing resources. When deploying applications to the cloud, a key step is to select the proper resource allocation that can satisfy the Quality-of-Service (QOS) requirement at a low cost. For AI-enabled health applications, the QoS requirement is usually the maximum model updating or inference latency allowed by the usage scenario.

To allow high flexibility in resource allocation, clouds offer their resources through a variety of types of allocation units, such as VMs, containers, and cloud functions. Within each type of allocation unit, there are also different resource configurations with different processors, memory, storage, and network. These large number of choices, however, make it very challenging even for cloud experts to accurately determine a good-performing and low-cost resource allocation. Moreover, the performance of a cloud application on a certain resource allocation also fluctuates randomly and significantly, which further complicates the determination of high QoS and low-cost resource allocation.

To handle the large number of resource allocation choices, prior work has proposed to employ automated search algorithms. In particular, Bayesian Optimization (BO) has been shown to be a potentially effective search algorithm for cloud resource allocation. The automated search typically starts with a group of resource allocation configurations, where the performance and cost of each configuration are recorded by executing the AI model on these resource configurations. These performance and cost data are then used as the training data to generate a regression model with BO. This regression model predicts a new resource allocation that potentially meets the QoS requirement with lower cost. The performance and cost of the new resource allocation are then evaluated and added to the training data to train a new regression model. This process is usually repeated until a large number of resource allocation configurations are explored. Finally, from the explore configurations, the lowest-cost configuration that satisfy the QoS is reported as the best configuration.

While this BO-based search made it possible to automatically determine a resource configuration, its accuracy and search cost (i.e., the number of configurations searched) are affected by cloud's random performance fluctuation. This BO-based search is only effective and accurate if the regression models built during the search are accurate. The accuracy of the regression models, in turn, relies on the reliability of the training data. However, due to cloud's random performance fluctuation, the performance data obtained from the search can have high errors, if the configurations are not properly evaluated. In fact, the original work that presented this BO-based search methodology executed the AI model on a resource configuration for only six trials to get the average performance. However, as the cloud performance may have up to 50% fluctuation, only six executions may produce an average performance that is significantly different than the real average performance of the AI model when it is executed for a much longer time. Indeed, the PI's prior work, and other cloud performance studies, have shown that several hundreds of trials may be required to obtain accurate performance in the cloud. The inaccurate performance data, in turn, can cause the BO-bases search to incorrectly report a final VM configuration that may miss the QoS requirement and/or have high cost.

To improve the accuracy of the regression models, prior work has proposed various solutions, such as enhancing the models with a set of micro-benchmarks or employing system-level performance metrics (e.g., CPU usage) as additional features. In Thrust 2, we proposed to employ one of his performance testing methodology, PT4Cloud, to enhance the BO-based search method, so that it can accurately determine the cost-efficiency resource configuration without the involvement of the developers of the health application developers.

As a preliminary study, we investigated if more reliable training data from PT4Cloud can improve the regression accurate of the first proposed BO-based methodology. That is, we used PT4Cloud's methodology to obtain the performance data for a cloud application running on a group of resource configurations in Amazon Web Service (AWS). These performance data were then used to build a regression model using BO (more accurately speaking, using the Gaussian Process as part of the BO). This regression model then predicted the performance of the cloud application when it was executing on a special type of VM, m5.xlarge in AWS. The predicted performance was compared with the ground truth performance to calculate is percentage error. The ground truth performance was obtained by executing the application on m5.xlarge for five weeks.

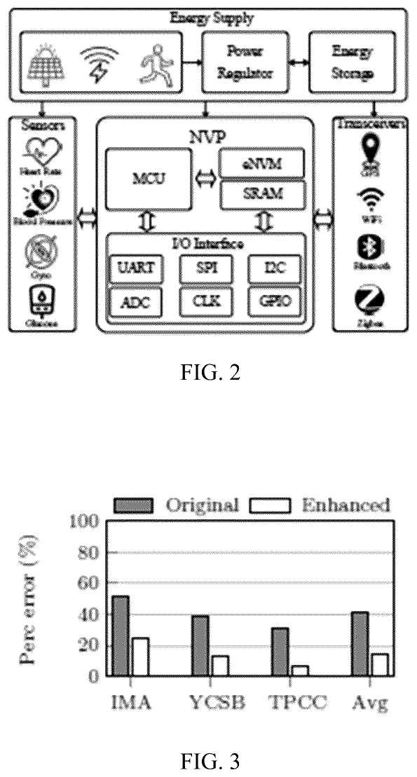

We evaluated three benchmarks from the CloudSuite, which were InMemory Analytics (IMA), YCSB, and TPC-C. For each benchmark, 1000 regression models were built, and the average error of these models are reported in FIG. 3. As a comparison, the errors of the original BO-based method using only 6 trials were also included in FIG. 3. As FIG. 3 shows, the more reliable performance data from PT4Cloud reduced the average prediction error from 40% to only 15%. In particular, in the case of TPC-C, the prediction error was reduced from 31% to only 7%. These preliminary results suggested that PT4Cloud could significantly improve the accuracy of the BO-based method, making it possible for an automated optimization framework without user-involvement.

### Proposed Research Tasks

Although our preliminary work showed that PT4Cloud cloud improve the regression model accuracy, additional research is still required to properly apply PT4Cloud to the BO search process. In this project, we propose two research tasks in Trust 2.

Task 2-1. In Task 2-1, we will first design and implement the optimization framework that combines PT4Cloud and BO-based search. More specially, PT4Cloud will be used to conduct performance tests to determine the cloud application's performance for each resource allocation. PT4Cloud will be redesigned into an automated testing harness that automatically executes the cloud application repeatedly until it deems that the performance result obtained from the executions has an error less than a user predefined maximum allowed error. For example, the tests in FIG. 3 used a max error of 3% (note that, this is the error for performance data used in training, not the regression model error).

The inputs into this optimization framework only include the AI model, example input/training data set for the model (such as the set of data used for training or a piece of data used to make an inference), and the number of training/inference requests per second. These inputs can be easily provided by the health application developers. For the maximum allowed error, we plan to start with 3%, which allows us to evaluate the framework with the most reliable performance data.

A potential risk of Task 2-1 is that the regression models may require more features than just the performance data and the information of the resource allocations. Therefore, we will consider including more features as suggested by prior work, such as CPU utilization and memory usage. To ensure minimal user-involvement, we will only consider features that can be automatically collected during the search. A second potential risk is mixing the resource configurations from IaaS (Infrastructure-as-a-Service), CaaS (Container-as-a-Service) and FaaS (Function-as-a-Service). Configurations from different types of clouds may have different behavior, and thus cannot be mixed when building the regression models. We may consider conducting separate searches for IaaS, CaaS, and FaaS, and then compare their best allocations to determine the actual best one.

Task 2-2. In Task 2-2, we seek to further reduce the search cost of our automated optimization framework. In particular, PT4Cloud may require hundreds of executions to obtain performance results with less than 3% error. However, for this cost-efficiency optimization, it may not be necessary to use performance data with such low errors. Indeed, the original BO-based search work stated that the regression model only needs to be accurate enough to distinguish the performance two resource allocations. Therefore, higher maximum allowed errors in PT4Cloud may still allow our optimization framework to determine the lowest-cost resource allocation with much fewer executions in PT4Cloud. Therefore, in Task 2-2, we will explore increasing the maximum allowed error gradually to find a max error that provides a good balance between optimization accuracy and search cost.

### Thrust 3: Edge-Enabled Optimizations for Sustainability and Cost-Efficiency

Edge servers, which act in the middle of sensing devices and clouds, are critical in supporting energy-efficiency and cost-efficiency for smart applications. For sensing devices, edge servers provide necessary computational power to support offloading of the relatively complex tasks, which work on either preprocessed (e.g., fused) sensor data or even raw sensor data from such devices, and thus improve their energy efficiency. On the other hand, for learning-intensive smart applications, edge servers can also assist the AI model inferences to reduce resource usages and resource cost in the cloud.

In this task, an automated holistic system optimization framework will be conducted to determine the energy-efficient and cost-efficient system design to support hundreds or thousands of users of an AI-enabled health application. We will explore loading some of the computations from the sensing devices and cloud to the edge servers to further reduce their energy usage and cost. As model training usually requires considerable computing resources, it needs to be performed on the cloud than offloaded to the edge servers. Therefore, in Thrust 3, we primarily focus on the energy/cost-efficient system design to support data preprocessing and AI model inference.

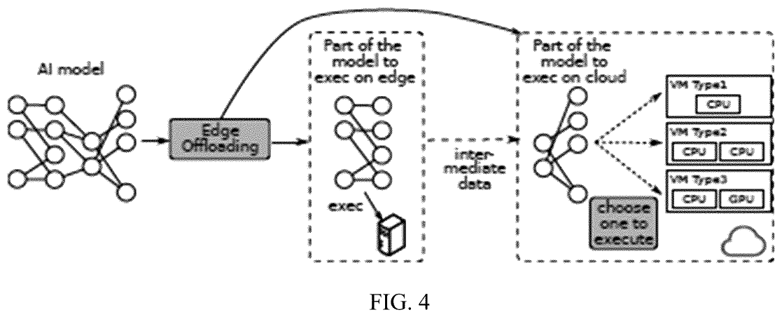

As stated above, two edge-offloading schemes are considered in Thrust 3. The first edge-offloading transfer some or all of the data preprocessing tasks to the edge servers from the sensing devices to reduce their energy consumption. The second edge-ooading is to partition an AI model between the edge and the cloud, as illustrated in FIG. 4. The main benefit of this edge-offloading is that it can reduce the resource demand (hence the cost) on the cloud. Note that, these two optimizations will eventually affect the design of the sensing devices and the resource allocation in the cloud. Therefore, the amount of computation offloaded to the edge servers needs to be co-optimized with the sensing devices and cloud deployments.

### Preliminary Study

As a preliminary study, we explored AI model partitioning with Google cloud functions on a low-power computer. This low power computer served as the edge server in this preliminary study. Two models were evaluated, which are the AlexNet and VGG models. For each model, we evaluated every partitioning of its layers. Table 1 gives the results of this preliminary study, including the inference latency when executing the models completely in the cloud (“w/o partitioning”), the lowest latency with partitioning, and the layer partition that gave the lowest latency.

As Table 1 shows, AI model partitioning can significantly affect the interference latency. In the case of VGG16, the latency was reduced from 31.89 seconds to 2.16 seconds. The reduction was primarily caused by the reduction in the time for transferring the data from the edge to the cloud. When using model partitioning, a much smaller set of data needed to be transferred to the cloud than no partitioning, and thus reducing the data transferring time. This reduction in data transfer also reduces the network component of the cloud usage cost. Moreover, as fewer layers were executed in the cloud, the computing resource usage cost in the cloud can also be reduced. For example, in the case of VGG16, the no-partitioning execution required 4.14 seconds in the cloud (including reading data and making inferences), whereas the best partitioning only required 0.24 seconds of execution time.

TABLE 1
Effectiveness of model partitioning.

| Model | w/o Partitioning | w Partitioning | Edge/Cloud Layer Split |
|---|---|---|---|
| AlexNet | 2.87 sec | 1.17 sec | 6 layers/7 layers |
| VGG | 31.89 sec | 2.16 sec | 24 layers/7 layers |

### Research Tasks

Based on the two offloading schemes, we propose two research tasks. The first task focuses on the improved sustainability of embedded sensing devices with edge offloading. The second task focuses on the co-optimization of the edge offloading and the cloud resource allocation.

Task 3-1: Enhanced Sustainability with Smart Edge Offoading.

Task 3-1 reuses the optimization framework from Thrust 1 to evaluate different data preprocessing offloading strategies. In this research task, we consider the close coordination between the sensing devices and edge servers to improve energy efficiency and latency. Here, by incorporating the optimizations for sensing devices developed in Research Thrust 1, the edge server can negotiate with sensing devices to ensure their sustainable operations via proper workload offloading while considering the latency requirements of applications. For instance, the edge servers may provide feedback or instructions to sensing devices for setting specific monitoring frequency or other functional requirements, based on the latency requirements of applications. Moreover, the edge server can interact with sensing devices to determine whether to offload/migrate workload base on the available energy, request processing latency, and resource availability. The sensing devices can also directly require the edge server to offload/migrate requests when their battery is low. We will explore the coordination between the sensing devices and the edge servers, and systematically evaluate what are the effects on energy efficiency for sensing devices as well as latency for the supported applications.

Task 3-2: Co-optimization of the edge and cloud. In Task 3-2, we will extend the automated optimization framework from Thrust 2 to conduct a design space exploration to determine the model partitioning and cloud resource allocation that satisfy the QoS requirement with the lowest cloud usage cost. That is, we will extend the BO-based search method to consider the number of offloaded layers as one of the configuration parameter to search. To use our optimization framework, the AI-enabled health application developers only need to provide the AI model, example input data used to make inferences, and the number of concurrent users/requests of the AI model inference.

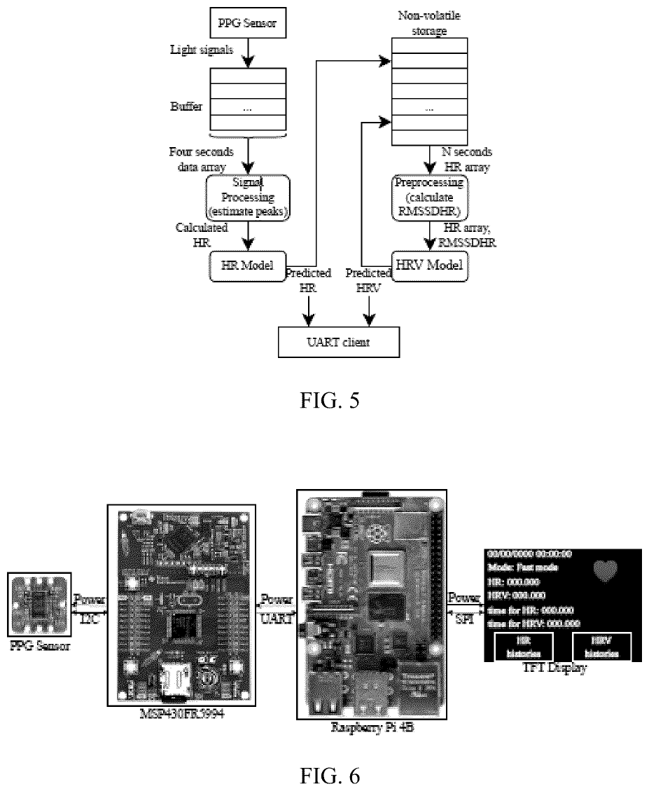

FIG. 5 gives the work flow proposed for Task 3-2. We will start with a set of randomly selected resource allocations (e.g., VM type and count) and randomly selected model partitions as the initial optimization configurations. Then for each VM configuration, we will conduct performance tests on the cloud (using PT4Cloud) and the edge server to determine the performance and the cloud usage cost of these configurations. With these performance and cost data, a regression model will be built following the methodology of BO, which will predict the next optimization (i.e., model partition and cloud resource allocation) to evaluate. The same process will be repeated for a number of iterations, and the configuration with the lowest cost will be reported by our optimization framework.

Similar to Thrust 2, the number of search iterations will be determined empirically. Additional features that can be automatically collected, such as the CPU and memory usage in the edge server and cloud, may also be added to the BO-based search. The optimization search will also be conducted separately on IaaS, CaaS, and FaaS clouds, and the most cost-efficiency configuration from the three types of clouds will be reported as the best by our framework.

### Evaluation Plan: Methodology and Validation

In addition to extensive simulations, computing systems composed of sensing devices (including both wearable and environmental sensors), edge servers, and clouds will be deployed for experimental studies to validate and evaluate the three proposed optimization frameworks in this project. Moreover, an AI powered glucose level prediction and diabetes management application will be used as the case study for evaluating the sustainability and cost-efficiency for the prototype system.

### Evaluation of Individual Research Tasks

The first set of evaluations examine individual research tasks. Here, various open-source AI models commonly utilized in smart health applications with di erent sensing devices, edge servers and cloud deployments will be employed in the evaluations.

Evaluation/Validation of Thrust 1: The main objective of Thrust 1 is to achieve sustainable and energy-efficient operations of the developed sensing devices under different sensing requirements, such as the required sensors, sensing frequencies, and data processing needs. Extensive simulations will be conducted based on the characteristics of sensors, computational units, and energy-harvesting components. Based on such simulation studies, the optimal design options will be explored to develop several prototype sensing devices. We will experimentally evaluate the performance and cost of such devices to validate the effectiveness of the design and optimization methodologies developed. In particular, the sustainability of wearable device prototypes powered by energy-harvesters with selected health sensors for the case study on glucose monitoring will be evaluated under different energy harvesting conditions.

Evaluation/Validation of Thrust 2: For Thrust 2, the main evaluation goal is to examine the cost of the cloud resource allocation scheme determined by our automated framework. More specifically, we will employ a group of open-source AI models with various complexities, such as AlexNet, VGG, blood pressure model, and fall detection. Our optimization framework will be applied to each AI model to generate a low-cost resource allocation. We will also conduct a manual search of the lowest-cost cloud resource allocation. Then the costs of the automatically determined and manually determine resource allocations will be compared and reported. The search cost of our optimization framework will also be reported. We will also compare with other state-of-the-art cloud resource allocation search algorithms, such as ChrreyPick and Arrow, to determine if our optimization framework can provide lower cost development by using more accurate training data.

Evaluation/Validation of Thrust 3: For Thrust 3, the main evaluation goal is to examine the effectiveness of the holistically-optimized system designs in terms of energy usage and cloud cost. Complete AI-enabled health applications, such as fall detection, which include both the sensors, data prepossessing, and AI models, will be used in this evaluation. In the experiments, Jetson TX2 boards, Raspberry PI boards, and several embedded devices will be used as the edge servers. For each application, our holistic optimization framework will be applied to each health application to determine the optimal workload distributions and offloading schemes for maximum sustainability and minimum cost. We will also conduct manual searches to determine the best system design for the sensing devices, edge offloading, and cloud deployments. Then the energy usage and cloud usage cost of the automatically and manually determined system designs will be compared to evaluate the effectiveness of our optimization frameworks.

### Case Study: AI-Enabled Glucose Prediction and Diabetes Management

In our prior work, we have conducted a study to predict future glucose levels with basic health behaviors and weight as features, where the work flow of the glucose level prediction technique has four steps: data collection, data pre-processing, model construction and prediction. Based on the sensing requirements and computation needs of the machine learning models and algorithms in the case study of AI-enabled glucose prediction and diabetes management applications, we will develop prototype wearable and environmental prototype sensing devices. These devices will integrate various environmental and wearable sensors and be designed/developed utilizing the proposed optimization framework with the focus on their sustainability and energy-harvesting features.

Then, a complete computing test-bed with the prototype sensing devices, edge servers, and deployed clouds will be established for the Smart Living Lab at UTHSCSA. Here, different software optimization packages and various offloading schemes will be implemented on such a test-bed based on the output of our proposed automated optimization framework. By exploiting the real health data collected from our previous studies on diabetes management, we will drive the emulated usage of the test-bed to collect experimental data on the energy consumption of sensing devices as well as the cost of operating the connected computing system with edge servers. Such experimental data will be used to validate and evaluate the effectiveness of the proposed optimization framework on improving the sustainability (i.e., energy-efficiency) and reducing the operating cost of real smart health applications (i.e., cost-efficiency). Note that, to protect privacy, this evaluation will only use simulated data, as well as previously collected anonymized data with no personal identifiers.

I. Adaptive Intelligent On-Device Monitoring of Heart Rate Variability with PPG.

Heart rate variability (HRV) is a critical vital sign that can predict a number of different diseases such as heart attack, arrhythmia, and stress. Traditionally, hospitals use electrocardiogram (ECG) devices to record the heart's bioelectrical signals which are converted to HRV values. Despite the high accuracy, this method is expensive and inconvenient. Recently, using photoplethysmography (PPG) sensors that collect reflective light signals has been adopted as a cost-effective alternative for measuring heart health. However, due to the sensitivity of PPG sensors, HRV estimation with PPG signals remains a challenging problem. To this end, this paper demonstrates an on-device lowcost HRV estimation system based on deep learning models with PPG sensors. The real-time HRV monitoring system is developed with a resource limited ultra-low-power microcontroller unit (MCU). In addition, it empowers the system with adaptive reconfiguration capability at run-time to improve energy efficiency and adapt to different demands. Moreover, the demo has a display to show HR and HRV in a real-time manner.

Heart rate variability (HRV) which measures the difference in time between successive heart beats is widely considered as one of the most important vital signs of body health [1]. HRV analysis has become an increasingly important diagnostic tool in cardiology as it shows relations to heart rate turbulence, maximal oxygen uptake, inflammatory response, and exercise capacity [2], [3]. While the traditionally used heart rate (HR) metric can indicate if a human is under stress, it cannot show how the human body reacts to the stress since HR is relatively stable, which fortunately can be shown by HRV since the time interval between consecutive heartbeats can vary. As a result, HRV monitoring system is indispensable for people who need real-time monitoring of heart activities.

Traditionally, hospitals use electrocardiogram (ECG) devices consisting of electrodes mounted on the human body to record the heart's electrical signals [4]. ECG devices can provide accurate and real-time HRV monitoring, which is the best choice for patients who need intensive care in hospitals. However, those ECG devices are heavy and not portable as they require cable connections. Recently, ECG technology has been integrated into Apple Watch [5], [6] for everyday HRV monitoring. Despite the capability of starting HRV monitoring anytime and anywhere, it has several limitations due to the special operation features of ECG. To start HRV monitoring with the ECG module, Apple Watch users are required to rest their arms on a table or on their lap and keep their fingers touching the Digital Crown (the button on Apple Watch), which is inconvenient and impossible for continuous long-time monitoring [7].

Alternatively, using photoplethysmography (PPG) sensors that collect reflective light signals appears to be a promising approach to measuring heart health as it is low-cost and more convenient than ECG devices [8]. Although PPG sensors cannot provide the R-R interval values which are the essential information for calculating HRV, they can extract the “peak”-“peak” interval values that can be interpreted as the cardiac R-R interval [9]. The location of a peak represents the time instant at which a heartbeat occurs. HRV computation requires accurate identification of the location of peaks in the PPG signal. However, due to the sensitivity of PPG sensors, HRV computation with PPG signals remains a challenging problem.

To address the problem, we design an adaptive intelligent on-device monitoring system of HRV with PPG sensors. This paper demonstrates our prototype deployed on an ultra-low-power microcontroller (MCU) and how it works by adaptively switching in different modes for different demands.

### Framework for Efficient PPG-Based Hrv Estimation

### A. Real-Time On-Device HRV Monitoring

Since the PPG sensor needs to be integrated into a lowpower wearable device with limited computing resources (low CPU frequency and small memory size), the implementation of real-time on-device HRV estimation needs to take the specifications of the ultra-low-power MCU into account. To fulfill the requirements of real-time, we propose the data pipeline as shown in FIG. 5.

Initially, the light signals of the PPG sensor will be stored in a buffer on MCU as the signals data array will be used to estimate peaks by a signal processing function. The size of the buffer is determined by the sampling rate times four seconds. For example, when the sampling rate of the sensor is set to 25 Hz, the buffer size is 100 (25×4) with each value being a four-byte floating number. Once we get the estimated peaks from signal processing, we get an approximately calculated HR. Then the calculated HR will be sent to the HR model as the input to predict HR. In our experiment, there is one HR being predicted and stored in the non-volatile memory every second. The predicted HR will be sent to a UART client to provide real-time monitoring. After every specific time interval (like the 30 s, 60 s, etc.), we will retrieve all historical HR to estimate an HRV during this period. The HR array will be sent to a preprocessing function to calculate one feature named RMSSDHR. The calculation for RMSSDHR is shown in Equation (1), where HRi denotes the ith HR, and N denotes the total number of HR in a given period (the period can be 30-300 seconds in our experiment). After that, we use the calculated RMSSDHR and the HR array as the input features to predict the HRV. The predicted HRV will be stored in the non-volatile memory and sent to the UART client for monitoring as well.

The data pipeline proposed above fully considers the memory and computing limitations of an ultra-low-power MCU. Non-volatile storage is used to avoid data loss when power is off. We only store light signals in the RAM to ensure enough memory space for HR signal processing, model inference, and HRV preprocessing.

### B. Adaptive Run-Time Reconfiguration

To implement run-time reconfiguration, we deploy three system configuration modes, which are designed for various demands, on the board as shown in Table 2. In these three modes, the framework will work under different deep learning models, sampling rate (SR), and MCU Digitally Controlled Oscillator (DCO) frequency. Thus as a result, with the preconfigurations, we can easily switch between different modes to meet different needs.

TABLE 2
Results under various system models.

| Mode | Model | DCO/MHz | SR/Hz |
|---|---|---|---|
| Fast mode | 121-10-10-1 | 8 | 25 |
| Energy saving mode | 121-10-10-1 | 1 | 12.5 |
| High accurate mode | 301-12-12-1 | 8 | 100 |

### Demonstration Setup

### Hardware Setup

FIG. 6 depicts the setup of the demonstration. The PPG sensor is MAXREFDES117 that provides reliable light signals and has noise filter units. The MSP430FR5994 development kit is one of the MSP430 families produced by Texas Instruments. The development kit contains everything needed to start developing on the ultra-low-power microcontroller platform, including on-device debug probe for programming, debugging, and energy measurements. The PPG sensor communicates with the MSP430FR5994 through the I2C bus. The MSP430FR5994 board connects to a Raspberry Pi through a micro-USB to get power and to a convenient UART communication. The Pi will decode the UART messages and display the monitoring results on a TFT LCD display with a resolution of 320*240.

II. Learning-Based Real-Time HRV Monitoring with PPG

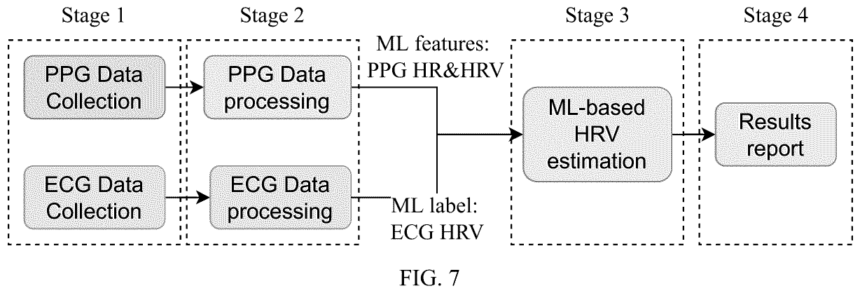

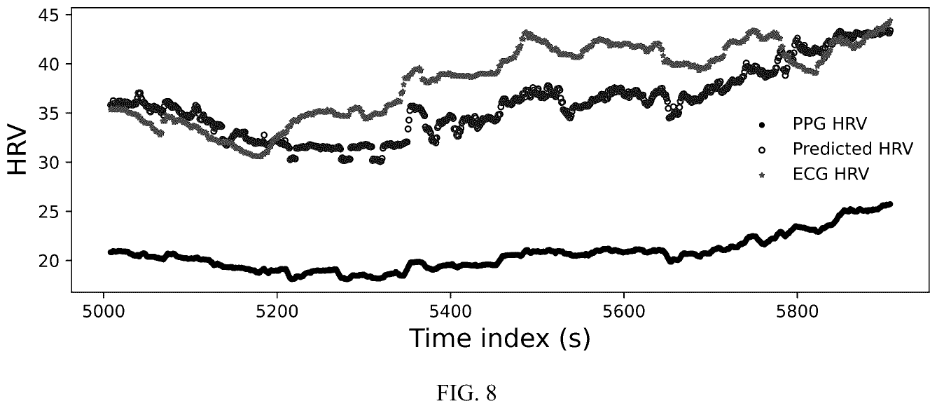

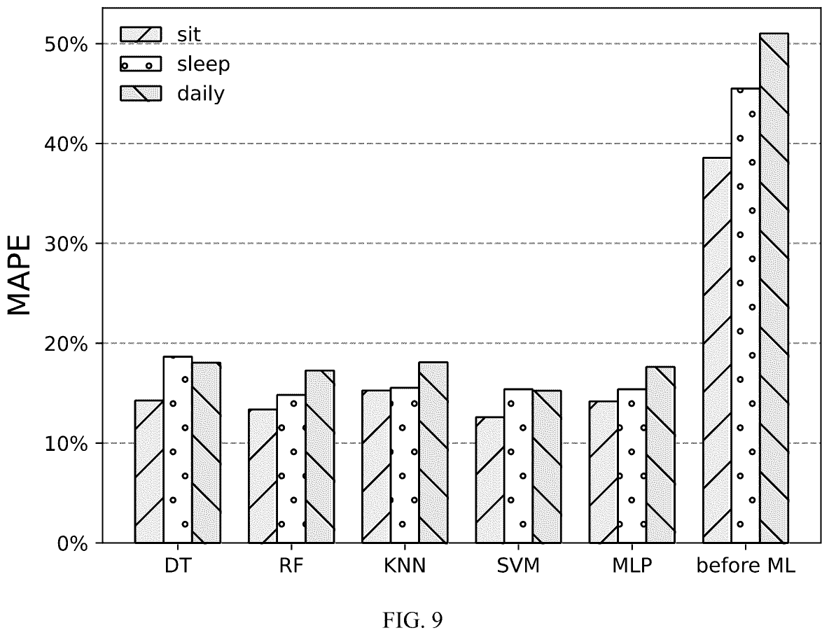

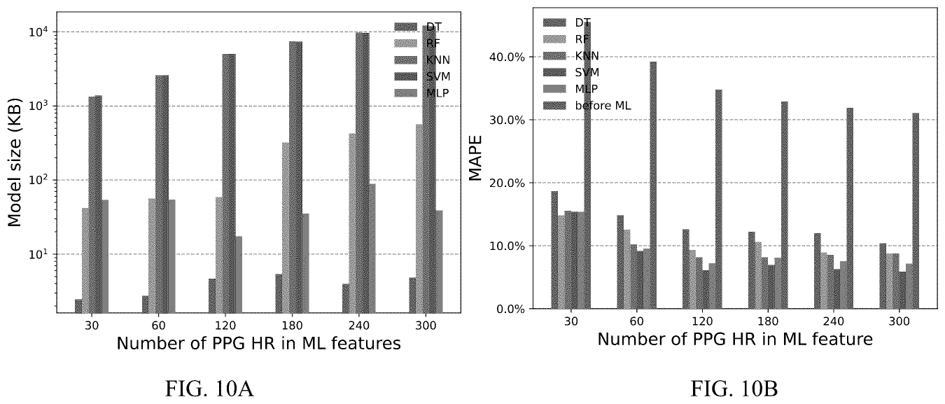

Heart rate variability (HRV) refers to the variation in the time interval between successive heartbeats, which is a critical health indicator of the autonomic nervous system. Many applications require monitoring HRV continuously such as arrhythmia detection and fatigue driving detection. The traditional approach to monitor HRV is using electrocardiogram (ECG) devices, which is expensive and inconvenient to operate in daily life. Photoplethysmography (PPG) sensor is an inexpensive optical measurement device that has been widely used in consumer wearable devices to monitor heart rates (HR), which can also be exploited to monitor HRV. FIG. 7 diagrams a system design and experiment flow including: Stage 1: PPG readings were collected from a PPG sensor, and synchronized ECG data were collected through an ECG device. Stage 2: processing algorithms were applied to reduce noise in the collected data and obtain the PPG HR, PPG HRV, and ECG HRV. Stage 3: for each instance, the most recent k PPG HR (1 HR per second), and the PPG HRV (calculated based on the most recent k heartbeat intervals) were passed to the machine learning (ML) model as features and the ECG HRV was passed as the label. Stage 4: the ML model estimated/predicted HRV was reported to the user and then compared with the ground truth—the ECG HRV to evaluate the performance.

The PPG sensor used is MAXREFDES117. It was installed on a fingertip and connected to the edge device—a Raspberry Pi 4B with 4 GB RAM. The ECG device is a portable 3-lead Holter TLC5007. We employed a widely used measure of HRV-Root Mean Square of Successive Differences (RMSSD). We collected PPG and ECG data for 2 hours in 3 different scenarios (sit, sleep, and daily). Daily data was collected when the subject was engaged in daily low-intensity activities like walking. ML model: 5 ML algorithms were implemented using python and scikit-learn on the edge device, including multiple layer perceptron (MLP), support vector machine (SVM), k nearest neighbor (KNN), decision tree (DT), and random forest (RF).

The mean average percentage error (MAPE) for ML predicted HRV is lower than the PPG HRV for all ML algorithms and scenarios. The daily scenario has a higher MAPE, which might be because there are more motion artifacts in the daily scenario. DT has the smallest model size (a few KB), while KNN and SVM have the largest model size (more than 10 MB). A larger k means more historical PPG HR in the ML features and it helps the ML model to be more accurate. All 5 ML models have a similar MAPE, and MLP is slightly more accurate than other algorithms.

The results from our preliminary study based on the limited PPG and ECG data validate the feasibility of the proposed HRV monitoring system, which can accurately estimate HRV in real-time based on PPG sensor data, powered by various ML algorithms. Motion artifacts have a large impact on the estimation accuracy, as the MAPE of the daily scenario is worse than the other two scenarios. ML algorithm: Compared to KNN and SVM, MLP, RF, and DT are more suitable for HRV estimation because they can estimate HRV more accurately with fewer resources.

III. On-Board Intelligent and Efficient Monitoring of HR/HRV with PPG and DNN Models

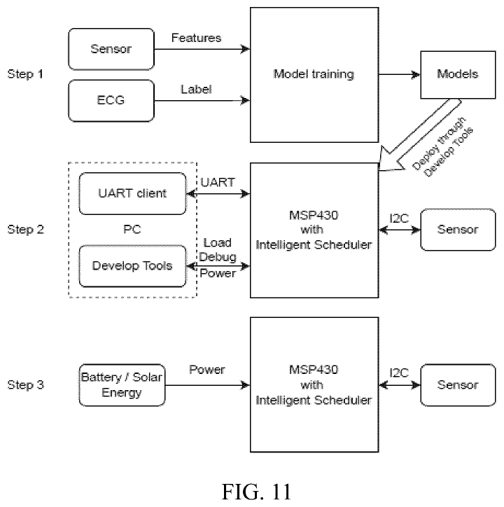

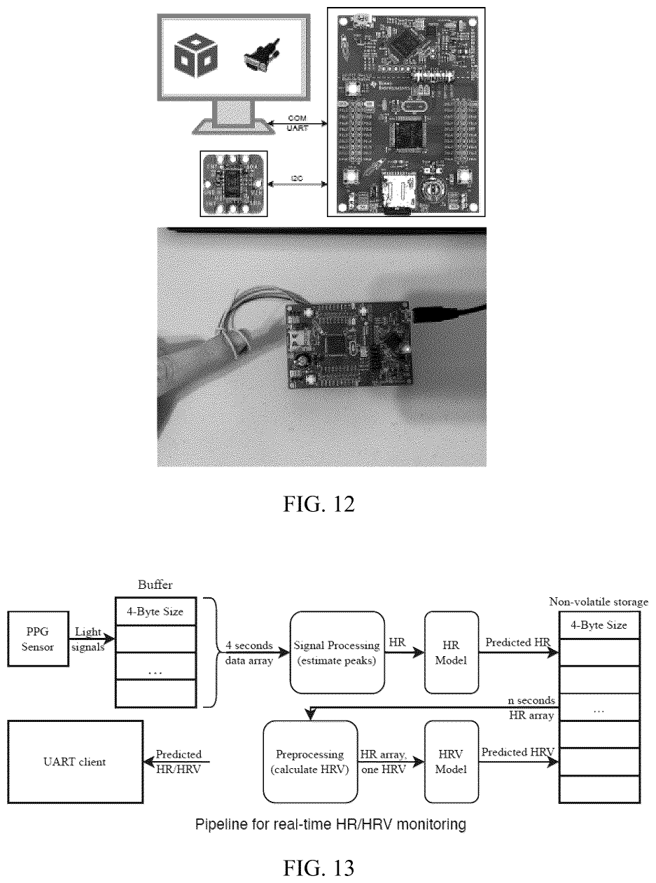

Although most modern smart wearable devices can report heart rate (HR) and heart rate variability (HRV), which are important health indicators, there are still challenges to accurately and efficiently monitor such indicators with wearable devices. In this work, with the focus on energy efficiency, we investigate intelligent methods to monitor HR/HRV using a PPG sensor on an ultra-low power development board with very limited computing power and memory capacity (see FIG. 11 and FIG. 12). Specifically: We designed a framework for learning-based HR/HRN monitoring with PPG and microcontroller; We investigated and identified the effective/efficient DNN models for PPG-based HR/HRV prediction; We analyzed performance/power characteristics of microcontroller with PPG for HR/HRV; We proposed adaptive run-time configurations for operation optimization and energy efficiency.

The frame work design includes model training and real-time HR/HRV estimation (FIG. 13). Model training includes (i) sensor: a PPG sensor integrated with MAX30102 module, (ii) Label: ECG device with the accurate electrical signals, (iii) Dataset: Both PPG and ECG monitor the signals simultaneously, (iv) Model: one HR DNN model and one HRV DNN model. The real-time HR/HRV estimation:

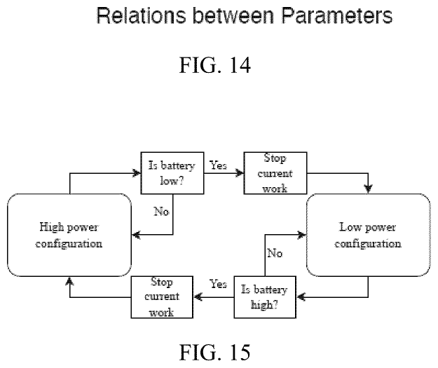

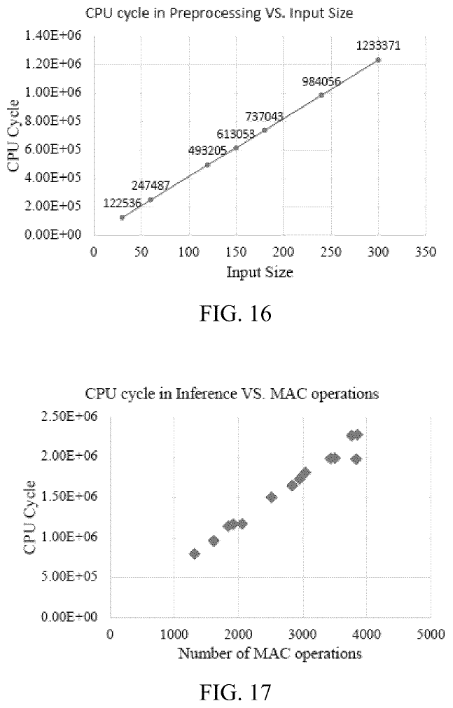

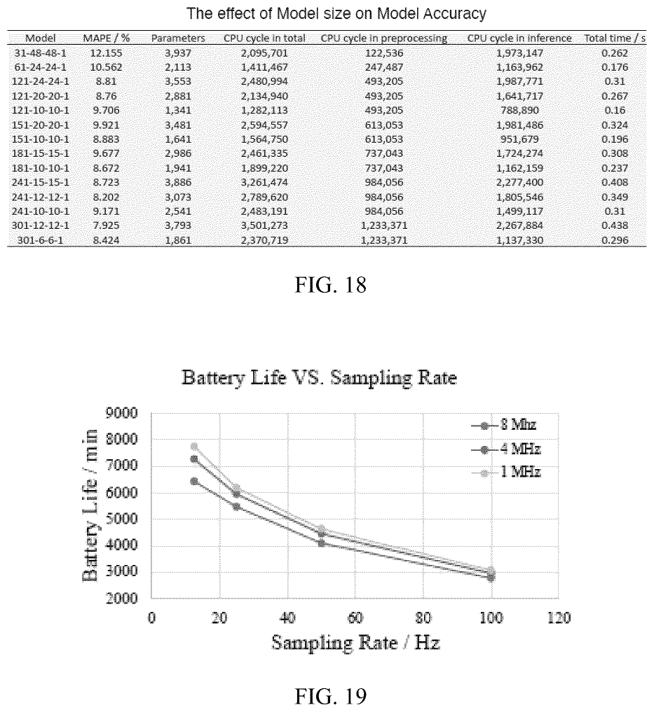

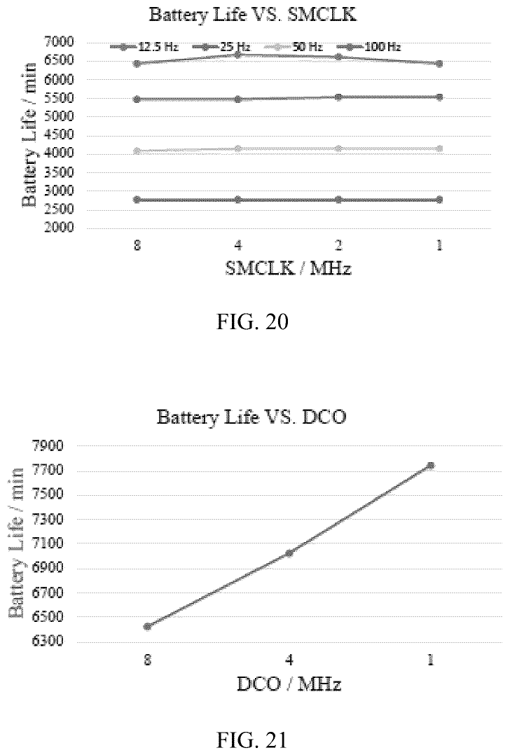

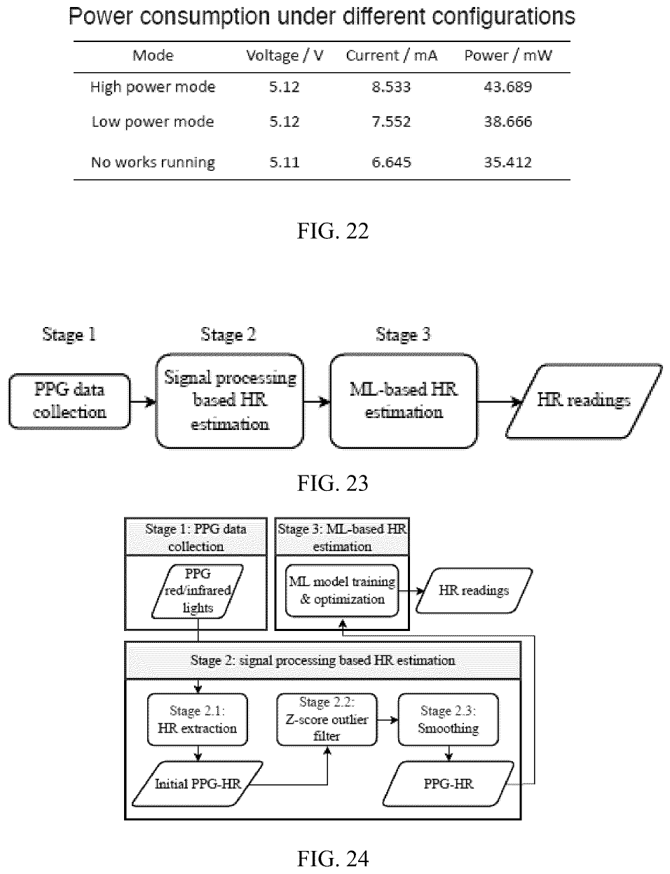

Power analysis considers model size and accuracy, inference time, battery life (limits, positive effects, negative effects, and unclear effects, FIG. 14. Runtime adaptive reconfigurations (FIG. 15) includes pre-stored configurations, stop when detecting battery level changes, and switch power mode by load different configurations. The results are illustrated in FIG. 16, FIG. 17, FIG. 18, FIG. 19, FIG. 20, FIG. 21, and FIG. 22.
IV. PPG-Based Heart Rate Estimation with Efficient Sensor Sampling and Learning Models

Recent studies showed that Photoplethysmography (PPG) sensors embedded in wearable devices can estimate heart rate (HR) with high accuracy. However, despite of prior research efforts, applying PPG sensor-based HR estimation to embedded devices still faces challenges due to the energy-intensive high frequency PPG sampling and the resource-intensive machine learning models. In this work, we aim to explore HR estimation techniques that are more suitable for lower-power and resource constrained embedded devices. More specifically, we seek to design techniques that could provide high-accuracy HR estimation with low-frequency PPG sampling, small model size, and fast inference time. First, we show that by combining signal processing and ML, it is possible to reduce the PPG sampling frequency from 125 Hz to only 25 Hz while providing higher HR estimation accuracy. This combination also helps to reduce the ML model feature size, leading to smaller models. Additionally, we present a comprehensive analysis on different ML models and feature sizes to compare their accuracy, model size, and inference time. The models explored include Decision Tree (DT), Random Forest (RF), K-nearest neighbor (KNN), Support vector machines (SVM), and Multi-layer perceptron (MLP). Experiments were conducted using both a popular existing data set and our self-collected data set. The experimental results show that our method by combining signal processing and ML had only 5% error for HR estimation using low-frequency PPG data. Moreover, our analysis showed that DT models with 10 to 20 input features usually have good accuracy, while are several magnitude smaller in model sizes and faster in inference time.

Heart rate (HR) is an important vital sign for the cardiovascular system and has been widely used as a biomarker for diagnostic and early prognostic of several diseases such as hypertension and heart failure. Besides the critical condition monitoring in the hospital setting, many applications also depend on continuously measured HR, such as fitness tracking, biometric identification, and frailty detection. Therefore, it is desirable to have a real-time HR monitoring system, which can conveniently provide accurate data in an effective manner to support such applications.

The traditional and reliable approach to continuously monitoring HR is to utilize electrocardiogram (ECG) devices, which are generally expensive and inconvenient to deploy for outpatients or other users to operate in a continuous manner. Besides ECG, Photoplethysmography (PPG), a handy sensor, when applied to the surface of the skin, like fingers, wrist, or earlobe, can utilize light signal to monitor changes in blood flow, which can be exploited to derive HR. Given its low cost and convenience, PPG sensors have been widely utilized as an inexpensive alternative to monitor HR in wearable embedded devices (such as smartwatches), which have limited energy and resources. However, as PPG is susceptible to motion artifacts (MA), existing PPG-based work that removes MA to detect HR typically has two limitations.

First, the classical method for extracting HR from PPG data is based on signal processing, which usually requires a relatively high sampling frequency (hundreds of Hz) and a complex algorithm to remove MA noises to achieve high accuracy. This high sampling frequency may incur high energy consumption, preventing these signal processing techniques from being applied to energy and resource-constrained devices. For example, a widely utilized data set for HR estimation is the IEEE Signal Processing Cup (ISPC) data set, which was proposed by Zhang et al., along with a signal processing-based algorithm that includes signal decomposition, sparse signal reconstruction, and spectrum peak tracking, to extract HR from PPG data (Zhang et al. IEEE Transactions on biomedical engineering, 62 (2): 522-31, 2014). The ISPC dataset contains PPG signals sampled at a high frequency of 125 Hz, which was widely adopted by other studies. However, Bhowmik et al. has reported that the PPG sensor on smartwatch sampling at 100 Hz consumed significantly more energy than 25 Hz (Bhowmik et al., 39th Annual International Conference of the IEEE Engineering in Medicine and Biology Society (EMBC), 109-12, 2017). Furthermore, certain signal processing techniques may require intricate algorithms to achieve high accuracy, which further worsens energy consumption.

Second, with the advancement of machine learning (ML), several studies exploited various machine learning models to remove MA noises and estimate HR. However, ML models, especially neural networks, usually are computationally intensive, limiting their application to embedded devices with limited resources. For example, Wittenberg et al. used complex deep learning models including convolutional neural network (CNN) and Gated recurrent unit (GRU) for PPG peaks detection (Wittenberg et al., Current Directions in Biomedical Engineering, 6 (3): 505-09, 2020). There were also studies that employed ML models such as K-means, Random Forest (RF), and Bayesian learning algorithm (Alqaraawi et al., Healthcare technology letters, 3 (2): 136-42, 2016; Bashar et al., 2019 International Conference on Electrical, Computer and Communication Engineering (ECCE), 1-5, 2019). Moreover, existing studies employ ML models usually employed a large number of features (i.e., PPG signals) to achieve high accuracy, which led to large models that may not fit in small embedded devices. To enable ML-based HR monitoring in a resource-constrained embedded device, research is needed to investigate feature dimension reduction to allow smaller ML models. There also needs exploration to determine the type of ML models that can provide accurate HR readings with low resource usage.

Described herein is the design of an HR monitoring solution using an off-the-shelf PPG sensor. The system is specially designed for resource-constrained devices and addresses the above limitations. To address the limitations, we combine signal processing and ML methods. That is, we first applied signal processing to generate rough HR estimations. Then these rough HRs are passed through a smaller ML model to generate more accurate HR estimations. On one hand, applying the ML model to signal-processing-generated HRs allowed us to sample PPG signals at only 25 Hz while achieving higher accuracy because ML models can be trained specifically to improve accuracy and remove MA with low-frequency samples. On the other hand, applying signal processing before ML eliminates the need for the ML model to directly take large numbers of PPG signals as inputs, reducing feature sizes and model sizes.

To address the ML model type issue of the second limitation, we compare the accuracy, model size, and inference time of five different ML models, including Decision Tree (DT), Random Forest (RF), K-nearest neighbor (KNN), Support Vector Machines (SVM) and Multi-layer Perceptron (MLP), and provide insights on which are more suitable for the resource-constrained device. Moreover, because existing data sets are usually too short for extensive evaluation, we also collected a new data set for HR study which is long enough for HR ML model training and testing with different features.

Experimental evaluations show that the system can achieve less than 5% mean average prediction errors (MAPE) for HR estimation with a PPG sampling rate of only 25 Hz. Moreover, our exploration results show that Decision Tree (DT) models usually could provide accurate estimation with a smaller model size of about 10 KB and a shorter inference time of less than 3 microseconds (μs). Contributions include: (i) Novel HR estimation methodologies that combined both signal processing and ML models, allowing high accuracy HR estimations with low-frequency PPG signals, fewer ML features, and smaller ML models. (ii) A systematic analysis of different ML model types and feature sizes to study their impact on the accuracy in HR estimation. This analysis showed that all considered ML models can provide about 5% MAPE for both the ISPC dataset and our collected dataset. (iii) A comprehensive analysis of the model size and inference time for different ML model types and configurations to study their suitability for the resource-constrained HR monitoring environment. This analysis found that DT can provide a good balance between accuracy, model size, and inference time.

A discussion of related work as well, as methodology and implementation, evaluation of results and conclusions are provided below.

### A. BACKGROUND AND CLOSELY RELATED WORK

In this section, we present the background and closely related work on HR estimation.

1. HR from ECG

The traditional medical device to measure HR is ECG. ECG records heart activity utilizing electrodes placed at certain skin spots on the human body and produces an electrocardiogram, which is a graph that shows the heart's electrical activity over time. An electrocardiogram contains the QRS complexes information, which is the most important waveform in an electrocardiogram that shows the spread of a stimulus through the ventricles. RR intervals can be derived from the QRS complex, which in turn, gives HR. More specifically, because the RR interval is the interval between heartbeats, the reciprocal of the RR interval is the HR. Although ECG can produce accurate HR, attaching electrodes to the human body makes it inconvenient to use.

2. HR from PPG-Signal Processing

Due to their small sizes, Photoplethysmography (PPG) sensors have become a popular replacement for ECG in HR monitoring. PPG uses light signals to monitor blood flow. Heartbeats cause periodical changes in the blood flow, which cause periodical changes in the reflected light received by the PPG sensor. Hence, periodical PPG light wave changes can be exploited to derive the heartbeat.

The main issue with PPG sensors is the noise in the signal, which is usually the result of motion artifacts (MA). The common method to remove MA and calculate HR is signal processing, which typically tracks the peaks of the PPG signals. Zhang et al. proposed an algorithm that consists of signal decomposition, sparse signal reconstruction, and spectrum peak tracking to extract HR from PPG signals in intensive physical activities environment. Their dataset, denoted as the IEEE Signal Processing Cup (ISPC) 2015 dataset, has been widely utilized for evaluating HR monitoring solutions. It includes PPG sensor data, accelerometer sensor data, and ECG data. In this ISPC dataset, the PPG sensor is installed on the wrist and the ground truth is a one-channel ECG. While in the work described herein, PPG is installed on fingertips and utilized a three-lead ECG to get ground truth HR. Additionally, each ISPC data recording lasts for only a few minutes, whereas each trace in our data lasts for about two hours.

3. C. HR from PPG-Machine Learning

Recently, ML has been found to be a promising method to remove MA from PPG signals and estimate HR. Prior studies utilized various ML algorithms to estimate HR. For example, Bashar et al. employed K-means clustering and Random Forest (RF) for HR estimation. The raw PPG signal was pre-processed with a 2nd order bandpass filter. Then the K-means clustering algorithm was used to identify noisy data and Random Forest regression was used to predict HR based on PPG and acceleration data. They used features extracted from PPG signals and examined adding features from accelerometers. Puranik and Morales estimated HR in real-time with an adaptive Neural Network filter and a postprocessing smoothing and median filter. Chang et al. came up with DeepHeart, an HR estimation approach that combines deep learning and spectrum analysis. The raw PPG signal was pre-processed by a three-order Butterworth bandpass filter. Then the PPG signal was sent to an ensemble model to remove noise. The ensemble model contains several deep learning models with convolutional layers. Biswas et al. proposed CorNET, a convolutional neural network with long short-term memory (LSTM) to estimate HR and perform biometric identification based on PPG signals. The raw PPG signal was pre-processed with a band-pass 4th order Butterworth filter and a normalizer. This method was estimated with leave-one-window-out validation on the ISPC dataset. Rocha et al. designed Binary CorNET, a binarized CorNET to estimate HR. All the above works used the ISPC dataset in their evaluation, which shows the popularity of the ISPC dataset. However, the ISPC dataset is insufficient for deep learning approaches since the available data for different activities is short and the total number of samples is limited. The ISPC dataset also used a high (125 Hz) sampling frequency, which incurs high energy usage.

Reiss et al. presented a CNN architecture for PPG-based HR estimation (Reiss et al., Proceedings of the 2018 ACM International Joint Conference and 2018 International Symposium on Pervasive and Ubiquitous Computing and Wearable Computers, 1283-92, 2018). They first preprocessed the PPG sensor with FFT and z-normalization, and then train and evaluate the CNN model using leave-one-session-out (LOSO) cross-validation with the ISPC dataset. In the experiment, they compared the proposed method with three classical signal processing methods and concluded that the performance of the CNN-based HR estimation is comparable with classical methods. Panwar et al. designed a convolutional neural network with LSTM to estimate HR and blood pressure based on PPG signals (IEEE Sensors Journal, 20 (17): 10000-011, 2020). They evaluated the model on the MIMIC dataset and showed the effectiveness of the neural model. The MIMIC dataset is collected from patients, while in this work, we focus on HR for healthy subjects.

Unlike prior studies which applied signal processing only for data preprocessing, our solution used signal processing first to generate a set of HR estimations, which are then further processed by ML models to improve estimation accuracy. By combining signal processing based and ML-based HR estimation, we can reduce both PPG sampling frequency and ML feature size while retaining high accuracy.

### B. LEARNING-ORIENTED EFFICIENT HR ESTIMATION WITH PPG DATA

In this section, we present the methodology for learning-oriented efficient HR estimation that combines signal processing and ML models using PPG data.

### 1. PPG-Based HR Monitoring System

FIG. 23 shows the architecture of our HR monitoring system, which has four stages. In stage 1, data collection modules collect the PPG signals. Here, a PPG sensor is attached to the subject's fingertip, which outputs red and infrared light signals. In stage 2, the signal processing module reduces noises in PPG data and generates rough estimations of HR for every second. In Stage 3, an ML module takes a sequence of rough HRs to get more accurate HR estimations. Finally, a report module reports the estimated HR readings to users. The rest of this section provides a detailed description of stages 2 and 3.

### 2. HR Estimation

FIG. 24 provides the detailed processing steps of our HR monitoring system, which are described as follows. (1) Signal Processing based HR Estimation (stage 2): The input data for stage 2 are time-series signals generated by PPG in the PPG data collection stage, which contains the red/infrared light signals reflected by blood under the skin. We employed a sampling rate of 25 Hz, i.e., 25 signal samples every second.
(a) Initial HR extraction (stage 2.1): The processing in step 2.1 takes the PPG light signals from the PPG data collection stage, finds the local peaks within the light sequence, and then calculated by counting the number of peaks to estimate HR. Currently, our signal processing algorithm converts 25 signals in a second to 4 HR readings for that second (denoted as initial PPG-HR).
(b) Z-score outlier filter (stage 2.2): Due to MA, there may be large noises in PPG signals, leading to outliers in the initial HR estimations from step 2.1. To eliminate these outliers, a Z-score outlier filter is applied.

The Z-score filter is a popular method used to find outliers. Given a data point, its z-score represents the distance (i.e., deviation) between its value and the mean of all data points, measured by multiples of standard deviation (std). For example, if a data point's z-score is 3, then the difference between this data point and the mean is 3×std. Therefore, if the z-score (i.e., distance) is larger than a threshold, then the data point can be viewed as an outlier. Here, we set the threshold z-score to 3 following common practice.

However, we cannot simply remove the outliers because HR is a time series that will be utilized as features by ML models. Here, deleting any values will possibly lose the time-related information. Therefore, we choose to revise the outliers' value to be the average of their two surrounding HR readings.
(c) Smoothing (stage 2.3): Even after the Z-score filter, there still could be abrupt peak or valley HR readings that are overly higher or lower than their surrounding HRs due to PPG signal noises, Therefore, to further reduce the PPG HR fluctuation, we applied additional data smoothing.

To smooth, we first take an average of the four HR readings within a second to convert them into one HR per second. This smoothing removes fluctuations of the HRs within a second. It also reduces the number of HRs to be input to the ML models in stage 3.

Moreover, normally, a person's HR does not change abruptly in one second and returns back in the next second. That is, an HR reading should not be significantly different from the HRs before and after it. Hence, it is possible to smooth the HR based on a specific upper and lower boundary to further restrict the PPG HR fluctuation. In our experiment, we set the boundary to 5%. That is, an HR reading can only be less than or equal to 5% above or below its predecessor. For HR readings that are more than 5% higher (or lower) than the previous value, their values are changed to be 5% higher (or lower) than the previous value. This 5% boundary is determined based on the fluctuation range observed from the more reliable ECG data.
(2) ML-based HR Estimation (stage 3): The rough HR estimations generated by signal processing (denoted as PPGHR) in stage 2 are passed to an ML model to generate a more accurate HR estimation. As our PPG sensor sampling rate was set at 25 Hz to preserve energy, the PPG-HRs from these samples may still contain large errors. The ML model is particularly trained to reduce errors due to the low sampling frequency.

More specifically, the ML model takes a sequence of the last k PPG-HR estimations to generate the current HR reading. Intuitively, the ML model uses these k PPG-HRs to assess the potential errors in them to produce a more accurate HR reading. We evaluated different values for k in our experiments. We also evaluated different types of ML models, including DT, RF, KNN, SVM, and MLP. These evaluation results are reported below.

### 3. Data Sets and Model Training

(1) Our Data Set and Model Training: As discussed previously, the features of our ML models are k last PPGHR estimations, i.e., the PPG-HR estimations from the last k seconds given one PPG-HR per second. The labels (i.e., ground truth HR) are obtained by ECG. During data collection, our subject wears the PPG monitoring system (described in Section III-A and ECG electrodes at the same time for data recording. Hence, the ECG can provide ground truth HR for corresponding PPG signals and PPG-HR. The subject engaged in three scenarios: sitting, sleeping, and conducting daily activities. The daily activity includes office working, walking, drinking water, etc. Each recording lasted for 2 hours.

The collected data sets are then partitioned into training and testing data sets, with a split of 80% and 20%, respectively. All ML models also went through random search based hyperparameter tuning to find the best model.
(2) ISPC Data Sets and Model Training: To show that the accuracy benefit from our combined signal processing and ML method is generic, we also applied our method to the popular ISPC dataset. This dataset provides raw PPG signals at 125 Hz along with ground truth HR readings.

Note that, since ISPC data sets used a different PPG sensor and different sampling rate, our signal processing steps cannot be applied to it. Therefore, we applied the signal processing steps in the original paper of the ISPC data set. Moreover, because the source code of the ISPC data set's signal processing is not publicly available, and the potential changes of the dataset after its publication, the HR estimation errors from our reproduced signal processing are not completely the same as those reported in the original paper.

### C. EXPERIMENTAL EVALUATION

### 1. Experiment Setup

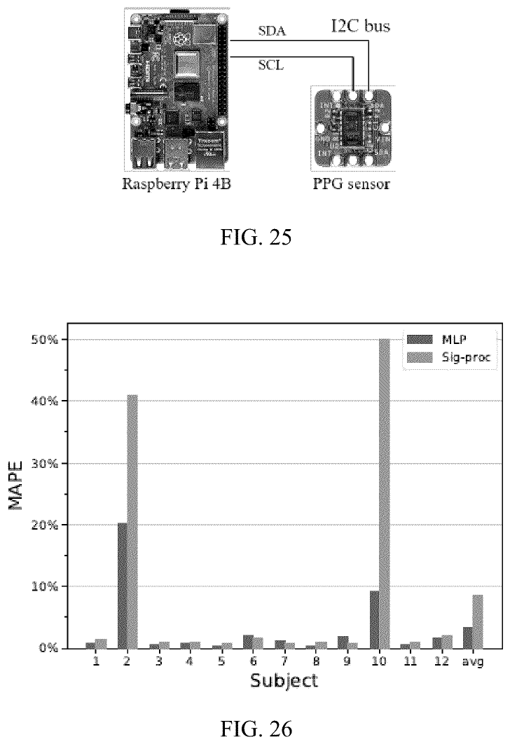

(1) ISPC dataset: The ISPC data set contains one channel of ECG data, 2 channels of PPG data, and 3-axis acceleration data. There are 12 healthy subjects and each of them is required to do a series of exercises to generate 5-minute long recordings with a PPG sampling rate of 125 Hz. Although there are two channels of PPG signals, we only used one channel that has lower noise for HR estimation for better reproduction accuracy. 80% of the data are used for training, and 20% are used for testing.
(2) Our dataset: Our data set contains one channel of PPG data and 3-lead ECG signals. The subject simultaneously wore the PPG sensor on the fingertip and ECG electrodes on the torso. The PPG sensor is connected to a Raspberry Pi 4B through an i2C bus to record the data, as shown in FIG. 25. The hardware components include: (1) a Raspberry Pi 4B with 4 GB RAM; (2) a Maxim MAXREFDES1171 HR monitor with a MAX30102 PPG sensor; and (3) a TLC5007 Dynamic ECG (not shown in the figure).
(3) ML Configurations: ML algorithms are implemented using Scikit-learn. We used the random search function RandomizedSearchCV in Scikit-learn to tune the model hyperparameters. The hyperparameters are: (1) For DT, the maximum depth of the tree ranges from 1 to 20. (2) For RF, the number of trees is between 1 to 30, and the maximum tree depth is between 3 to 7. (3) For KNN, the number of neighbors is between 1 to 30, and the distance can be Manhattan or Euclidean. (4) For SVM, the kernel may be among RBF, sigmoid and polynomial, and regularization (i.e., C) may be between 0.00001 to 10. 5) For MLP, there are 3 hidden layers with 2 to 15 neurons in each layer. The activation function can be relu or tanh. The L2 regularization hyper-parameter alpha search range is from 0.00001 to 10.

### 2. Heart Rate Estimation Results

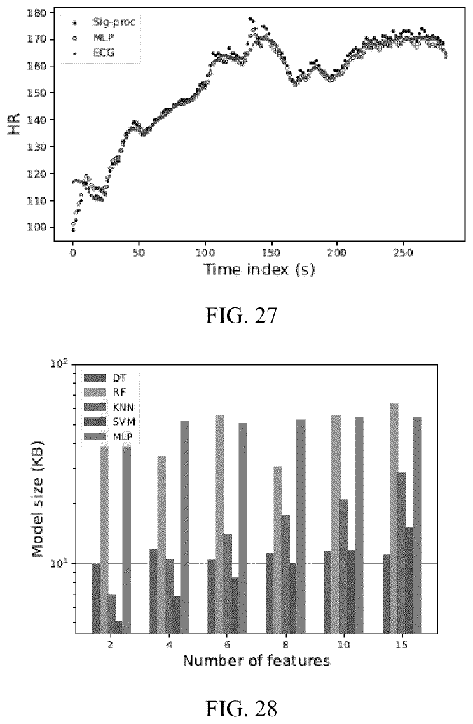

(1) HR Estimation Accuracy for ISPC Data Set: FIG. 26 gives the HR estimation errors for the ISPC data sets, including the MAPEs of our method that combines signal processing and MLP model, and the MAPEs of the signal-processing-only method. Note that, the MLP model here has two features (i.e., two PPG-HRs as features). As FIG. 26 shows, our combined method usually had lower errors than the signal-processing only method. On average, our method had only 3.33% error, which is 5.17% lower than the signal-processing-only method. To further compare the accuracy differences, FIG. 27 gives the HR estimations of our combined method, the signal processing-only method, along with the ECG ground truth. As FIG. 27 shows, our HR estimations are more close to ECG readings, and less fluctuating than those from the signal processing-only method, suggesting that the MLP model in our method could indeed reduce the random errors from the HRs generated by signal processing.

FIG. 26 also indicates that, for most subjects, the HR estimation errors are below 2%, suggesting that PPG-based HR monitoring can have high accuracy. The only two exceptions are subject 2 and subject 10, where the PPG signals were more noisy and random, likely due to worse MA effects during data collection. The same issue was also observed by other studies. Nonetheless, even for subjects 2 and 10, our combined method is still significantly more accurate than the signal-processing-only approach.
(2) ML Model Exploration for ISPC Data Set: Besides MLP with two features, we also explored ML models and feature sizes to search for a configuration that can provide good accuracy with low model size and short inference time. Table 2 gives the MAPEs for different ML models and feature sizes (i.e., PPG-HR input sizes). Note that, the MAPEs in Table 2 is the average MAPEs for all 12 subjects for each configuration along with the standard deviation. As Table 2 shows, while all configurations have similar low errors, RF with 15 features has the lowest average MAPE at 2.62%, followed closely by DT with 15 features at 2.76%.

TABLE 2
MAPE and SD for different number of features when estimating ISPC HR (MAPE ± SD)

| | Number of features | | | | | |
|---|---|---|---|---|---|---|
| | 2 | 4 | 6 | 8 | 10 | 15 |
| DT | 3.86% ± 5.26% | 3.77% ± 4.91% | 3.58% ± 4.14% | 3.4% ± 3.64% | 3.41% ± 3.53% | 2.76% ± 1.89% |
| RF | 3.14% ± 3.45% | 3.29% ± 3.4% | 2.9% ± 2.37% | 2.79% ± 2.08% | 2.7% ± 1.87% | 2.62% ± 1.91% |
| KNN | 2.89% ± 2.6% | 2.89% ± 2.2% | 2.9% ± 1.87% | 3.08% ± 1.75% | 3.55% ± 2.33% | 3.69% ± 1.94% |
| SVM | 3.81% ± 2.5% | 5.14% ± 6.18% | 4.71% ± 5.49% | 3.83% ± 2.2% | 5.11% ± 5.41% | 4.06% ± 2.49% |
| MLP | 3.33% ± 5.86% | 3.26% ± 5.2% | 3.38% ± 5.49% | 4.53% ± 5.78% | 3.4% ± 4.41% | 4.81% ± 4.46% |

FIG. 28 gives the average model sizes for all subjects with different ML configurations, which shows that DT usually has smaller model sizes. While KNN and SVM have smaller model sizes when the number of features is small, due to their algorithmic philosophy, their model sizes grow rapidly when the number of features increases.

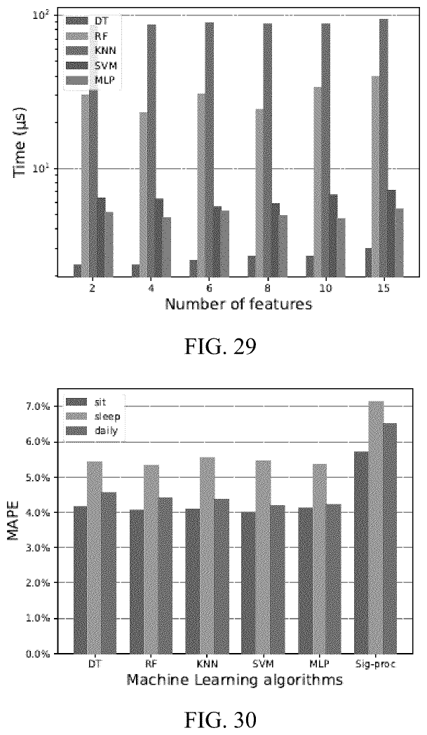

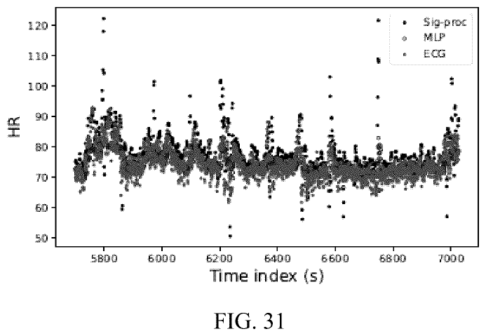

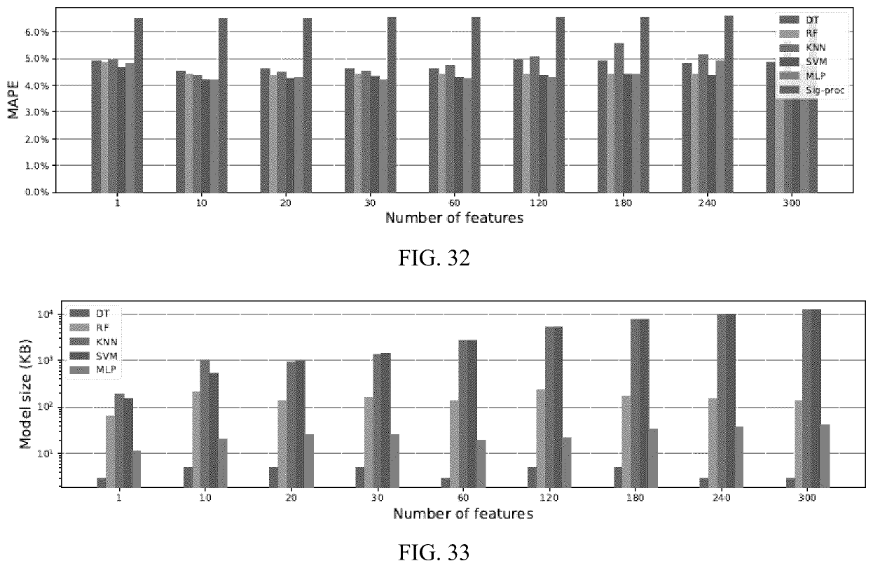

FIG. 29 gives the average model inference time for all subjects with different ML configurations, which shows that DT usually has shorter inference time. The inference time did not increase much when the number of features increased from 2 to 15. Considering the lower error from DT, these results suggest that DT with 15 features is a better configuration that provides both high accuracy, low model size, and short inference time.
(3) HR estimation Accuracy for Our Data Set: FIG. 30 gives the HR estimation error using our data set. Note that, the ML models here have 10 features (i.e., 10 PPG-HRs as features). As FIG. 30 shows, our combined method with either DT, RF, KNN, SVM, and MLP models always has lower error than the signal-processing-only model (Sig-proc) for all activity scenarios. Moreover, the errors of our method are always below 6%, showing that our method is highly accurate. FIG. 31 further gives the HR traces for our method with an MLP model, the signal-processing-only method, and the ECG ground truth. Similar to the ISPC evaluation results, HRs from our combined method in FIG. 31 are usually more close to ECG readings. Our HR estimations are also less fluctuating than those from the signal-processing-only method, suggesting that our method could reduce the random noises in the PPG signals when the PPG signal is only at 25 Hz.
(4) ML Model Exploration for Our Data Set: Again, we also explored ML models and feature sizes using our data set to search for a configuration with good accuracy, low model size, and short inference time. Since our data duration is longer than ISPC data, we took advantage of this and explored up to 300 features to further investigate how much historical data is preferable as ML input. FIG. 32 gives the MAPEs for different ML models and feature sizes (i.e., PPGHR input sizes) for the daily activity scenario. We can see from FIG. 32, that the errors for all types of ML models are similarly low when there are more than 10 features, suggesting that 10 to 20 features could be enough for our data set.

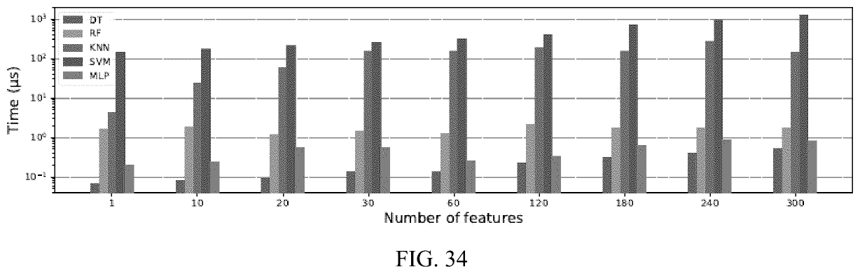

FIG. 33 gives the model sizes of different ML configurations, which shows that DT models are consistently the lowest. FIG. 34 provides the model inference time with different ML configurations, which reveals that DT models are consistently the fastest. All the inference times increased slightly when the number of features increased from 1 to 300, due to more features and bigger models. Considering the lower error from DT, these results suggest that DT with 10 to 20 features is a better configuration that provides high accuracy with a low model size and fast inference time.

While Photoplethysmography (PPG) sensors could provide accurate HR estimations, applying these sensors to embedded devices is still challenging due to the high-frequency PPG sampling, which has high power consumption, and the complex machine-learning models, which are too computational-intensive and large for small-embedded devices. It is shown that combining signal processing and ML could significantly reduce PPG sampling frequency while providing high HR estimation accuracy. The experimental results showed that our method that combined signal processing and ML had only 5% error for HR estimation using low-frequency PPG data. This combination also reduces the ML model's feature size, leading to smaller models. Additionally, we also conducted a comprehensive analysis of different ML models and feature sizes to compare their accuracy, model size, and inference time. Our analysis showed that DT models with 10 to 20 features usually have good accuracy while being several magnitude smaller in model sizes and faster in inference time.

1. A method for estimating heart rate (HR) with high accuracy comprising:
(i) obtaining signal from one or more photoplethysmography (PPG) sensor in contact with a subject,
(ii) combining (a) signal processing generating first HR estimations and (b) passing the first HR estimations through machine learning (ML) model generating second accurate HR estimations reducing the PPG sampling frequency to about or less than 25 Hz and providing higher HR estimation accuracy achieving less than 5% mean average prediction errors (MAPE).

2. The method of claim 1, wherein ML is selected from Decision Tree (DT), Random Forest (RF), K-nearest neighbor (KNN), Support vector machines (SVM), and Multi-layer perceptron (MLP).

3. The method of claim 1, wherein the ML is DT.

4. The method of claim 3, wherein the DT has 10 to 20 input features.

5. The method of claim 3, wherein DT models have a model size of about or less than 10 KB and a shorter inference time of less than 3 microseconds (μs).

6. The method of claim 1, further comprising placing the PPG on the skin of a subject.

7. The method of claim 6, wherein the PPG is placed on a subject's finger, wrist, or earlobe.

8. A wearable device for estimating heart rate (HR) with high accuracy configured to (i) obtain signal from one or more photoplethysmography (PPG) sensor in contact with a subject, (ii) combine signal processing and machine learning (ML) reducing the PPG sampling frequency to about or less than 25 Hz and providing higher HR estimation accuracy.

9. The wearable device of claim 8, wherein ML is selected from Decision Tree (DT), Random Forest (RF), K-nearest neighbor (KNN), Support vector machines (SVM), and Multi-layer perceptron (MLP).

10. The wearable device of claim 8, wherein the ML is DT.

11. The wearable device of claim 10, wherein the DT has 10 to 20 input features.

12. The wearable device of claim 8, configured to contact the PPG with skin of a subject.

13. The wearable device of claim 8, wherein the wearable device is configured to be placed on a subject's finger, wrist, or earlobe when in use.
